(19) United States
(12) Patent Application Publication
(10) Pub. No.: US 2024/0148917 A1
(43) Pub. Date: May 9, 2024

(54) PNA PROBES FOR PRETARGETED IMAGING AND THERAPY

(71) Applicant: Zytox Therapeutics AB, Johanneshov (SE)

(72) Inventors: Amelie Eriksson Karlström, Bromma (SE); Kristina Westerlund, Hägersten (SE); Hanna Tano, Stockholm (SE)

(21) Appl. No.: 18/249,192

(22) PCT Filed: Oct. 18, 2021
(86) PCT No.: PCT/EP2021/078854

§ 371 Date: Apr. 14, 2023

(30) Foreign Application Priority Data
Oct. 16, 2020 (SE) 2051204-2

(51) Int. Cl. A61K 51/08; A61K 47/60; A61K 47/64; A61K 47/66; A61K 49/00; A61P 35/00

(52) U.S. Cl. CPC A61K 51/088; A61K 47/60; A61K 47/64; A61K 47/665; A61K 49/0002; A61P 35/00

(57) ABSTRACT

The invention relates to a kit for targeting of a diagnostic or therapeutic agent to a target site comprising: (a) a first conjugate comprising (i) a targeting moiety capable of binding selectively to the target site; and (ii) a first hybridization probe moiety comprising a PNA oligomer; and (b) a second conjugate comprising (i) a second hybridization probe moiety comprising a complementary PNA oligomer; and (ii) a diagnostic agent or a therapeutic agent moiety; wherein the length of the complementary PNA oligomer in the second hybridization probe moiety is not more than 14 bases. The invention further relates to methods for delivering a diagnostic or therapeutic agent to a target site in mammals, as well as methods for the diagnosis or treatment of medical conditions, such as e.g. cancer, in mammals.

### TECHNICAL FIELD

The invention relates to a kit for targeting of a diagnostic or therapeutic agent to a target site comprising: (a) a first conjugate comprising (i) a targeting moiety capable of binding selectively to the target site; and (ii) a first hybridization probe moiety comprising a PNA oligomer; and (b) a second conjugate comprising (i) a second hybridization probe moiety comprising a complementary PNA oligomer; and (ii) a diagnostic agent or a therapeutic agent moiety. The invention further relates to methods for delivering a diagnostic or therapeutic agent to a target site in mammals, as well as methods for the diagnosis or treatment of medical conditions, such as e.g. cancer, in mammals.

### BACKGROUND ART

Affibody® molecules are small (molecular weight 7 kDa) engineered scaffold proteins, which can be selected to bind with high affinity to a broad spectrum of biomolecules (Stahl 2017) and belong to a class of engineered scaffold proteins with potential for cancer diagnostics and therapy (Weidle 2013). Affibody® molecules are well suited as radionuclide imaging probes due to their small size, high affinity and selectivity to cancer-associated targets (Stahl 2017). Affibody® molecules can easily be recombinantly produced in high yields in prokaryotes. Affibody-based imaging probes for epidermal growth factor receptor (EGFR or HER1), human epidermal growth factor receptor type 2 (HER2), human epidermal growth factor receptor type 3 (HER3), platelet-derived growth factor receptor β (PDGFRβ), insulin-like growth factor-1 receptor (IGF-1R), vascular endothelial growth factor receptor 2 (VEGFR2) and programmed death ligand 1 (PD-L1) have demonstrated very promising features in preclinical experiments (Tolmachev 2020). Furthermore, excellent imaging of HER2 has been demonstrated in clinics (Sorensen 2014; Sorensen 2016).

Targeting of HER2 using monoclonal antibodies and antibody-drug conjugates extends the survival of breast and gastroesophageal cancer patients, but an onset of resistance to such therapies is inevitable despite preserved HER2 expression (Kreutzfeldt 2020; Garcia-Alonso 2020). A HER2-targeted radionuclide therapy might be a solution in this case. However, the mainstream approach to targeted radionuclide therapy, the use of radiolabeled monoclonal antibodies, is inefficient in solid tumors due their long residence in circulation causing excessive irradiation of bone marrow (Larson 2015). Direct application of Affibody® molecules for radionuclide therapy is complicated due to high renal reabsorption and long retention of activity in the case of radiometal labels (Fortin 2008). Common methods applied for reduction of renal uptake of radiolabeled proteins and peptides turned out to be inefficient for Affibody® molecules (Altai, 2013; Garousi 2020).

A solution to the problem of high renal reabsorption of radiolabeled Affibody® molecules is applying pretargeting, a methodology that separates the acts of molecular recognition of cancer-associated abnormalities and radionuclide delivery (Frampas 2013, Altai 2017 JNM). In pretargeting, a target-specific primary agent coupled to a recognition tag is injected to localize in the tumor. After clearance of the primary agent from blood, a radiolabeled secondary probe with high affinity to the recognition tag is injected. A low uptake of the secondary probe in kidneys is critical for successful affibody-based pretargeted therapy. Affibody® molecules are attractive candidates for primary probes because they clear rapidly from blood and are slowly internalized by cancer cells (Wållberg 2008).

After evaluation of different approaches (Altai 2016; Honarvar 2016), hybridization of complementary peptide nucleic acid (PNA) probes was selected for affibody-based pretargeting because it provided the best retention of activity in tumors. PNA is a class of synthetic DNA analogues capable of Watson-Crick base-pairing (Egholm 1993; Nielsen 1994). The PNA backbone is built up of repeating N-(2-aminoethyl)-glycine units connected by amide bonds, and purine and pyrimidine nucleobases are connected to this scaffold via carboxymethyl linkers. PNAs are resistant to degradation by nucleases and proteases and have shown excellent stability in human blood serum (Demidov 1994). They are non-immunogenic and have low general toxicity. The molecular design of the first-generation of the primary agent Z_{HER2:342}-SR-HP1 and the secondary probe HP2 was successful, as it provided high affinity and specificity of PNA hybridization, specific accumulation of the primary probes in tumors, and efficient specific delivery of radiometals (Westerlund 2015, Honarvar 2016). Labeling of HP2 with ¹⁷⁷Lu (Altai 2017 NMB, Westerlund 2018), ¹¹¹In (Westerlund 2015, Honarvar 2016) and ⁶⁸Ga (Vorobyeva 2018) resulted in appreciably higher uptake in tumors than in kidneys, although the renal uptake was the highest among normal tissues.

Experimental therapy using the Z_{HER2:342}-SR-HP1/[¹⁷⁷Lu]Lu-HP2 pretargeting system significantly increased the median survival of mice bearing HER2-expressing xenografts (66 days for treated group compared to 32 days for [¹⁷⁷Lu]Lu-HP2 only) without observable bone marrow and renal toxicity (Westerlund 2018). However, a further increase in the ratio of absorbed dose to tumor compared to doses to normal tissues, first and foremost kidneys, is necessary to obtain a curative effect of such treatment.

A possible optimization parameter is the length of the secondary probe. Reduction of the length would reduce the hydrodynamic radius of the probe, which might facilitate both its extravasation and diffusion in tumor interstitium improving both localization in tumors and uniformity of distribution inside the tumor. There are, however, apparent risks associated with reduction of the secondary probe size. First, reduction of the number of nucleobases might decrease the strength of hybridization with the primary probe. Second, modification of the base composition might affect off-target interactions resulting in elevated uptake in normal tissues. For example, tiny structural changes associated with the substitution of ¹⁷⁷Lu by ¹¹¹In or ⁶⁸Ga resulted in significant differences in renal uptake (Vorobyeva 2018), or uptake in blood, liver and bone (Altai 2017 NMB). The biodistribution is further dependent not only on the number and nature of nucleobases but also on their order in a PNA sequence. The scrambling of nucleobases in ^{99m}Tc-labeled antisense PNA binding to mRNA encoding MYC protein resulted in more than two-fold decrease of uptake in normal tissues (Rao 2003, Mather 2004). Thus, experimental in vivo studies are required to evaluate if the second generation secondary probes would provide a better biodistribution and dosimetry profile.

### BRIEF DESCRIPTION OF THE DRAWINGS

FIG. 1: SPR sensorgrams of single-cycle kinetic titration of HP16, HP17, HP18 and HP19 binding to immobilized Z_{HER2:342}-SR-HP15. Each PNA probe was injected at the concentrations 22.6, 45.3, 90.6, 181.25 and 362.5 nM.

FIG. 2a: Normalized melting temperature curves shown for the three PNA-hybridization complexes: HP15:HP16; HP15:HP17; and HP15:HP18.

FIG. 2b: Normalized melting temperature curve for the HP15:HP19 PNA-hybridization complex.

FIG. 3: (A) In vitro binding specificity of the primary agent (A) [¹⁷⁷Lu]Lu-Z_{HER2:342}-SR-HP15; (B) [¹⁷⁷Lu]Lu-HP16; (C) [¹⁷⁷Lu]Lu-HP17; and (D) [¹⁷⁷Lu]Lu-HP18, on SKOV3 and BT474. The data are presented as an average value from 3 samples±SD.

FIG. 4: Biodistribution of: (A) [¹⁷⁷Lu]Lu-HP16; (B) [¹⁷⁷Lu]Lu-HP17; and (C) [¹⁷⁷Lu]Lu-HP18, at 4 h after injection in female Balb/c nu/nu mice bearing SKOV3 xenografts with and without pre-injection of Z_{HER2:342}-SR-HP15. The uptake was expressed as % ID/g and presented as an average±SD (n=5).

FIG. 5: SPECT/CT imaging of HER2-expressing SKOV3 xenografts for pretargeting using: (A) [¹⁷⁷Lu]Lu-HP16; (B) [¹⁷⁷Lu]Lu-HP17; (C) [¹⁷⁷Lu]Lu-HP18; and (D) [¹⁷⁷Lu]Lu-HP2, 4 h after injection.

FIG. 6: Time-activity plots for: (A) [¹⁷⁷Lu]Lu-HP16; (B) [¹⁷⁷Lu]Lu-HP17; (C) [¹⁷⁷Lu]Lu-HP18; and (D) [¹⁷⁷Lu]Lu-HP2. Non-decay corrected data for both kidney and tumor was used.

### DISCLOSURE OF THE INVENTION

Disclosed herein are second generation hybridization probes, the primary HP15 and a set of secondary probes: HP16 (9-mer PNA), HP17 (12-mer PNA), HP18 (15-mer PNA) and HP19 (6-mer PNA) (Table 1). As shown in the Examples, probes carrying a DOTA chelator were designed, synthesized, characterized in vitro, and labelled with ¹⁷⁷Lu. In vitro pretargeting was studied in HER2-expressing SKOV3 and BT474 cell lines. The biodistribution profile of these novel probes was evaluated in BALB/C nu/nu mice bearing SKOV3 xenografts and compared to the previously studied [¹⁷⁷Lu]Lu-HP2.

Characterization by SPR and UV spectroscopy confirmed the formation of high affinity duplexes between HP15 and the secondary probes HP16, HP17, HP18, and HP19, with the affinity correlating with the length of the complementary PNA sequences. The three tested (HP16, HP17, HP18) PNA-based probes bound specifically to HER2-expressing cells in vitro with high affinity (11-12 pM). In vivo studies demonstrated HER2-specific uptake of all [¹⁷⁷Lu]Lu-labeled probes in HER2-expressing xenografts. The ratio of cumulated radioactivity in the tumor to the radioactivity in kidneys was dependent on the secondary probe's size and decreased with an increased number of nucleobases. The shortest PNA probe tested in vivo, [¹⁷⁷Lu]Lu-HP16, showed the highest tumor-to-kidney ratio and is the most promising secondary probe for affibody-mediated tumor pretargeting.

In a first aspect, the invention provides a kit for targeting of a diagnostic or therapeutic agent to a target site comprising (a) a first conjugate comprising (i) a targeting moiety capable of binding selectively to the target site; and (ii) a first hybridization probe moiety comprising a first PNA oligomer; and (b) a second conjugate comprising (i) a second hybridization probe moiety comprising a second PNA oligomer complementary to the first PNA oligomer; and (ii) a diagnostic agent or a therapeutic agent moiety; wherein the length of the second PNA oligomer in the second hybridization probe moiety is not more than 14 bases.

In the present context, the term “kit” should be understood as meaning or including a composition of chemical and/or biological compounds, such as a pharmaceutical composition.

According to the invention, the use of a second PNA oligomer which has a length of not more than 14 bases will result in an improved (increased) tumor-to-non-tumor tissue ratio for the diagnostic agent or therapeutic agent, when a first and second conjugate are administered by a pretargeting protocol to a mammal having a tumor, compared to an otherwise comparable situation where the diagnostic agent or therapeutic agent is administered without the pretargeting protocol i.e. with the targeting moiety coupled to the therapeutic/diagnostic moiety in the same entity.

The relevant non-tumor tissue will vary depending on the specific application area. For instance, in therapeutic applications, there generally is a non-tumor tissue in which the therapeutic agent causes toxicity limiting the doses that can be administered to a patient without unacceptable side effects, called dose-limiting tissue. The invention may result in a ratio of tumor-to-dose-limiting tissue that it at least 2-fold (preferably at least 2.5-fold, more preferably at least 3-fold, most preferably at least 3.5-fold) compared to otherwise comparable situation but without the pretargeting protocol i.e. with the targeting moiety coupled to the therapeutic/diagnostic moiety in the same entity. The particular dose-limiting non-tumor tissue will in turn depend on factors such as the nature of the targeting moiety and the therapeutic agent moiety. The non-tumor tissue can for instance be kidney, bone, liver or stomach, preferably kidney. In diagnostic applications, it is desirable to minimize interfering background signal for the diagnostic marker from tissue surrounding the tumor to be investigated, especially blood. Thus, the non-tumor tissue may also be blood.

In the present context, the term “pretargeting protocol” means that the first conjugate is administered to the mammal before administration of the second conjugate, so that association between the first and second conjugate takes place in vivo.

With regard to the first and second PNA oligomers, the term “complementary” PNA oligomers refers to PNA oligomers that can form a double-stranded structure by matching base pairs. Preferably, there is a complete complementarity between the two PNA strands, that is each base is across from its opposite. However, the degree of complementarity could be less than complete (100%), provided that the two probes can hybridize to form a structured duplex.

The degree of complementarity between two nucleic acid strands may vary, from complete complementarity (each nucleotide is across from its opposite) to no complementarity (each nucleotide is not across from its opposite)

Preferably, the said target site resides on a mammalian, including human, protein which is expressed on the surface of a tumor cell. Preferably, the target protein is overexpressed on tumor cell surfaces with no or little expression on normal healthy tissues. For instance, the protein can be selected from the group consisting of epidermal growth factor receptor (EGFR), human epidermal growth factor receptor 2 (HER2), human epidermal growth factor receptor 3 (HER3), insulin-like growth factor 1 receptor (IGF1R), carbonic anhydrase IX (CAIX), platelet-derived growth factor receptor β (PDGFR-β), nectin-4, cluster of differentiation 38 (CD38), cluster of differentiation 33 (CD33), cluster of differentiation 30 (CD30), cluster of differentiation 22 (CD22), and cluster of differentiation 79b (CD79b) (see Table 7). Preferably, the target protein is a human protein. Preferably, the said target site resides on human epidermal growth factor receptor 2 (HER2).

The said targeting moiety is preferably selected from the group consisting of:
antibodies, such as monoclonal antibodies;
antibody fragments, such as single-domain antibodies (sdAb; nanobodies), single-chain variable fragments (scFv), antigen-binding fragments (Fab), diabodies (cf. Holliger et al. 1993), or minibodies (cf. Hu et al. 1996);
engineered scaffold proteins, such as Affibody® molecules; and
peptides, such as a tumor-targeting peptide selected from a combinatorial library (cf. Liu et al., 2017), or a peptide analog based on an endogenous peptide binding to a G-protein coupled receptor (GPCR) (cf. Hauser, 2017; and Moody, 2018).

In a preferred aspect of the invention, the said targeting moiety is an Affibody® molecule, such as an Affibody® molecule targeting HER2, for example the molecule designated Z_{HER2:342}, or other similar molecules as disclosed in Orlova et al. 2006 and in WO 2005/003156.

The term “Affibody® molecule” refers to a small engineered scaffold protein, which can be selected to bind with high affinity to a broad spectrum of biomolecules. Affibody molecules are small (58-amino-acid residue) protein domains derived from one of the IgG-binding domains (the Z-domain) of staphylococcal protein A. Affibody molecules have a three-helix bundle structure that can been used as scaffold for construction of combinatorial libraries, from which Affibody molecule variants that target desired molecules can be selected. For a review, see e.g. Tolmachev 2020.

Preferably, the first hybridization probe moiety is covalently attached to the targeting moiety. Preferably, the first hybridization probe moiety is conjugated to the targeting moiety via sortase A-mediated ligation, as described in e.g. Westerlund, 2015. The Staphylococcus aureus sortase A is a transpeptidase that attaches surface proteins to the cell wall; it cleaves between the Gly and Thr of an LPXTG motif and catalyzes the formation of an amide bond between the carboxyl-group of threonine and the amino-group of the cell-wall peptidoglycan. The recognition motif (LPXTG) is added to the C-terminus of a protein of interest, while a single glycine or an oligo-glycine peptide having a free N-terminus is added to the second molecule to be ligated. Upon addition of sortase to the molecules, the two molecules are covalently linked through a native peptide bond.

Optionally, the one or both of the hybridization probe moieties comprise a moiety which improves solubility. The said moiety may be a solubilizing moiety, such as a PEG-based linker comprising e.g. (PEG)₂, (PEG)₄, (PEG)₆, etc. Preferably, the PEG-based linker is a linker comprising 2-[2-(2-aminoethoxy)ethoxy]acetic acid (AEEA). Alternatively, or in addition, the linker comprises charged or polar amino acids, such as Glu, Asp, Ser, Thr, Lys, or Arg. Preferably, the solubilizing moiety comprises AEEA.

According to the invention, the length of the first PNA oligomer in the first hybridization probe moiety is preferably at least the length of the second PNA oligomer in the second hybridization probe moiety, and not more than 15 bases. More preferably, the length of said PNA oligomer in the first hybridization probe moiety is 15 bases. A preferred 15-base sequence is the sequence c-c-t-g-g-t-g-t-t-g-a-t-g-a-t (SEQ ID NO: 3). In preferred aspects of the invention, the said first hybridization probe moiety has the structure
G-S-S-c-c-t-g-g-t-g-t-t-g-a-t-g-a-t-E-K-[Linker]-E-NH₂
   or
G-S-S-c-c-t-g-g-t-g-t-t-g-a-t-g-a-t-E-K([Chelator])-[Linker]-E-NH₂
   wherein G, S, E and K represent the amino acids Gly, Ser, Glu and Lys, respectively. In one aspect, the first hybridization probe moiety has the structure
G-S-S-c-c-t-g-g-t-g-t-t-g-a-t-g-a-t-E-K(DOTA)-AEEA-E-NH₂
   wherein DOTA is the chelator 1,4,7,10-tetraazacyclododecane-1,4,7,10-tetraacetic acid and AEEA is 2-[2-(2-aminoethoxy)ethoxy]acetic acid.

According to the invention, the length of the second PNA oligomer in the second hybridization probe moiety is between 5 and 14 bases, such as preferably 8-14 or 9-14 bases, or more preferably 9-12 bases, inclusive. The second PNA oligomer has a sequence which is complementary to, and thus hybridizing with, the sequence of the first PNA oligomer in the first hybridization probe moiety.

In one preferred aspect, the length of the second PNA oligomer is 9 bases. A preferred 9-base sequence is the sequence a-a-c-a-c-c-a-g-g (SEQ ID NO: 4). In one preferred aspect, the second hybridization probe moiety has the structure
[Chelator]-[Linker]-S-S-a-a-c-a-c-c-a-g-g-E-E-Y—NH₂
   wherein S, E and Y represent the amino acids Ser, Glu and Tyr, respectively. In one aspect, the second hybridization probe moiety has the structure
DOTA-AEEA-S-S-a-a-c-a-c-c-a-g-g-E-E-Y—NH₂
   wherein DOTA is the chelator 1,4,7,10-tetraazacyclododecane-1,4,7,10-tetraacetic acid and AEEA is 2-[2-(2-aminoethoxy)ethoxy]acetic acid.

In another preferred aspect, the length of the second PNA oligomer is 12 bases. A preferred 12-base sequence is the sequence a-t-c-a-a-c-a-c-c-a-g-g (SEQ ID NO: 5). In one preferred aspect, the second hybridization probe moiety has the structure
[Chelator]-[Linker]-S-S-a-t-c-a-a-c-a-c-c-a-g-g-E-E-Y—NH₂
   wherein S, E and Y represent the amino acids Ser, Glu and Tyr, respectively. In one aspect, the second hybridization probe moiety has the structure
DOTA-AEEA-S-S-a-t-c-a-a-c-a-c-c-a-g-g-E-E-Y—NH₂
   wherein DOTA is the chelator 1,4,7,10-tetraazacyclododecane-1,4,7,10-tetraacetic acid and AEEA is 2-[2-(2-aminoethoxy)ethoxy]acetic acid.

According to the invention, the first and second hybridization probe moieties may comprise PNA sequences which carry modifications, like substitutions, small deletions, insertions, or inversions, and still maintain the biological activity of the PNA sequences shown as SEQ ID NOS: 1-3. Preferably, such modifications do not involve more than 1, 2, or 3 of the bases in any one of SEQ ID NOS: 1-3.

For example, the 9-base sequence shown as SEQ ID NO: 4 may be modified with 1 or 2 substitutions or deletions, resulting in a sequence having at least 75%, or at least 85% identity with SEQ ID NO: 4.

Further, the 12-base sequence shown as SEQ ID NO: 5 may be modified with 1, 2 or 3 substitutions or deletions, resulting in a sequence having at least 75%, at least 83%, or at least 91% identity with SEQ ID NO: 5.

The 15-base sequence shown as SEQ ID NO: 3 may be modified with 1, 2 or 3 substitutions or deletions, resulting in a sequence having at least 80%, at least 86%, or at least 93% identity with SEQ ID NO: 3.

It will be understood that the modified PNA oligomer has a sequence which is complementary to, and thus hybridizing with, the sequence of the PNA oligomer in the other hybridization probe moiety. As mentioned above, the degree of complementarity could be less than complete (100%). Consequently, there could be 1 or 2 mismatches in such a duplex, while the hybridized conjugates would still be useful according to the invention.

When the second conjugate comprises a therapeutic agent moiety, the therapeutic agent may be a radionuclide, or a cytotoxic drug suitable for use in antibody-drug conjugates

(ADC) or other drug-comprising conjugates. (For a review, see e.g. Shim, 2020.) For instance, a said cytotoxic drug can be selected from calicheamicin, auristatins such as monomethyl auristatin E/F (MMAE/F), and maytansinoids, such as maytansine derivatives DM0-DM4. Preferably, the therapeutic agent is a radionuclide.

When the therapeutic agent is a radionuclide, the radionuclide may preferably be selected from the group consisting of Lutetium-177 (¹⁷⁷Lu), Yttrium-90 (⁹⁰Y), Bismuth-212 (²¹²Bi), Bismuth-213 (²¹³Bi), Astatine-211 (²¹¹At), Actinium-255 (²⁵⁵Ac), Copper-67 (⁶⁷Cu), Gallium-67 (⁶⁷Ga), and Rhenium-186 (¹⁸⁶Re). More preferably, the radionuclide is Lutetium-177 (¹⁷⁷Lu).

When the second conjugate comprises a diagnostic agent moiety, the said diagnostic agent preferably generates a signal that is detectable by a method selected from the group consisting of positron emission tomography (PET), single photon emission computed tomography (SPECT), and optical imaging.

In one preferred aspect, the said diagnostic agent is a radionuclide. When the method for signal detection is SPECT, the radionuclide is preferably selected from the group consisting of Technetium-99m (^{99m}Tc), Indium-111 (¹¹¹In), Lutetium-177 (¹⁷⁷Lu), Iodine-123 (¹²³I) Iodine-125 (¹²⁵I) Gallium-67 (⁶⁷Ga), and Copper-67 (⁶⁷Cu). More preferably, the radionuclide is Indium-111 (¹¹¹In).

When the method is PET, the radionuclide is preferably selected from the group consisting of Gallium-68 (⁶⁸Ga), Fluorine-18 (¹⁸F), Iodine-122 (¹²²I), Iodine-124 (¹²⁴I), and Copper-64 (⁶⁴Cu). More preferably, the radionuclide is Gallium-68 (⁶⁸Ga).

When the method for signal detection is optical imaging, the said diagnostic agent is preferably a fluorescent dye selected from the group consisting of cyanine dyes, porphyrin derivatives, phthalocyanines, squaraine derivatives, xanthenes, Alexa analogues, and BODIPY analogues.

When the diagnostic agent or therapeutic agent is a radionuclide, the second hybridization probe moiety preferably comprises a chelator for radiometal complexing. The said chelator can be selected from the group consisting of:
1,4,7,10-tetraazacyclododecane-1,4,7,10-tetraacetic acid (DOTA),
1,4,7-triazacyclononane-1,4,7-triacetic acid (NOTA),
1,4,8,11-tetraazacyclododecane-1,4,8,11-tetraacetic acid (TETA),
1,4,7-triazacyclononane, 1-glutaric acid-4,7-acetic acid (NODAGA), and diethylenetriaminepentaacetic acid (DTPA). In one preferred aspect of the invention, the chelator is DOTA.

However, it will be understood that some radionuclides, such as Fluorine-18 (¹⁸F), Iodine-122 (¹²²I), Iodine-123 (¹²³I) Iodine-124 (¹²⁴I), and Iodine-125 (¹²⁵I) can be conjugated to hybridization probes without the aid of a chelator, such as by fluorination or iodination.

When the diagnostic agent or therapeutic agent is other than a radionuclide, such as a cytotoxic drug, the diagnostic agent or therapeutic agent is preferably covalently attached to the second hybridization probe moiety. For instance, the second hybridization probe moiety can be conjugated to the diagnostic agent or therapeutic agent moiety via sortase A-mediated ligation, as described in e.g. Westerlund, 2015.

Optionally, the kit according to the invention comprises a clearing agent, capable of removing circulating primary conjugate which is not bound at the target site. As disclosed in e.g. the international patent publication WO 96/40245, the clearing agent may be an anti-idiotypic antibody or antigen-binding antibody fragment.

In a further aspect, the invention provides a pharmaceutical composition comprising a kit as defined above. The invention further provides the use of a kit or a pharmaceutical composition as defined above for (a) targeting of a diagnostic or therapeutic agent to a target site; and/or (b) diagnosis, prognosis or treatment of a medical condition in a mammal, including a human.

In yet another aspect, the invention provides a method for delivering a diagnostic or therapeutic agent to a target site in a mammal, including humans, said method comprising:
(a) administering to said mammal a first conjugate comprising
   (i) a targeting moiety which binds selectively to the target site; and
   (ii) a first hybridization probe moiety comprising a first PNA oligomer;
(b) optionally, administering to said mammal a clearing agent, and allowing said clearing agent to clear non-localized first conjugate from circulation; and
(c) administering to said mammal a second conjugate comprising
   (i) a second hybridization probe moiety comprising a second PNA oligomer complementary to the first PNA oligomer; and
   (ii) a diagnostic agent or a therapeutic agent moiety;
wherein the length of the second PNA oligomer in the second hybridization probe moiety is not more than 14 bases, and wherein said second PNA oligomer binds to the first PNA oligomer in the first conjugate, thereby targeting the diagnostic agent or therapeutic agent moiety to the target site.

In a preferred aspect, the method as defined above implies a method for the diagnosis, prognosis or treatment of a medical condition in a mammal, including humans.

According to the above-identified methods, the said first and second conjugate, as well as the optional clearing agent, are preferably as defined above in the context of the disclosed kit according to the invention.

In yet another aspect, the invention provides a diagnostic or therapeutic conjugate for use in a method for delivering a diagnostic or therapeutic agent to a target site in a mammal, including humans, said method comprising:
(a) administering to said mammal a targeting conjugate comprising
   (i) a targeting moiety which binds selectively to the target site; and
   (ii) a first hybridization probe moiety comprising a first PNA oligomer;
(b) optionally, administering to said mammal a clearing agent, and allowing said clearing agent to clear non-localized targeting conjugate from circulation; and
(c) administering said diagnostic or therapeutic conjugate to said mammal, wherein said diagnostic or therapeutic conjugate comprises:
   (i) a second hybridization probe moiety comprising a second PNA oligomer complementary to the first PNA oligomer; and
   (ii) a diagnostic agent or a therapeutic agent moiety;
      and wherein the length of the second PNA oligomer in the second hybridization probe moiety is not more than 14 bases, and wherein said second PNA oligomer binds to the first PNA oligomer in the first conjugate, thereby targeting the diagnostic agent or therapeutic agent moiety to the target site.

In a preferred aspect, the method as defined above implies a method for the diagnosis, prognosis or treatment of a medical condition in a mammal, including humans.

According to the above-identified methods, the said targeting conjugate is preferably as the first conjugate defined above in the context of the disclosed kit according to the invention and the said diagnostic or therapeutic conjugate is preferably as the second conjugate defined above in the context of the disclosed kit according to the invention.

According to the above-identified methods, the optional clearing agent is preferably as defined above in the context of the disclosed kit according to the invention.

In the methods and uses according to the invention, the first and second conjugates can be administered intravenously, intraarterially, intrapleural, intraperitoneally, intrathecally, subcutaneously or by perfusion.

In the methods and uses according to the invention, the said medical condition may be selected from the group consisting of cancer, infectious diseases, inflammatory diseases, and autoimmune diseases. Preferably, the medical condition is cancer.

In one preferred aspect, the said medical condition is cancer capable of forming solid tumors, said cancer selected from the group consisting of breast cancer, prostate cancer, lung cancer, head- and neck cancer, gastric cancer, and colon cancer. In such cases, the said target site preferably resides on a mammalian or, more preferably, a human protein selected from the group consisting of epidermal growth factor receptor (EGFR), human epidermal growth factor receptor 2 (HER2), human epidermal growth factor receptor 3 (HER3), insulin-like growth factor 1 receptor (IGF1R), carbonic anhydrase IX (CAIX), platelet-derived growth factor receptor β (PDGFR-β), and nectin-4 (see Table 7). In one preferred aspect, the method of the invention is a method for internal detection or treatment of a HER2-expressing carcinoma, in particular a carcinoma associated with breast cancer or gastroesophageal cancer.

In another preferred aspect of the invention, the medical condition is hematological cancer selected from the group consisting of melanoma, leukemia, and myeloma. In such cases, the said target site resides on a mammalian or, more preferably, a human protein selected from the group consisting of: cluster of differentiation 38 (CD38), cluster of differentiation 33 (CD33), cluster of differentiation 30 (CD30), cluster of differentiation 22 (CD22), and cluster of differentiation 79b (CD79b) (see Table 7).

### Numbered Items of the Invention

Embodiments of the invention include the items summarized in the following, non-exclusive, list:
1. A kit for targeting of a diagnostic or therapeutic agent to a target site comprising:
   (a) a first conjugate comprising
      (i) a targeting moiety capable of binding selectively to the target site; and
      (ii) a first hybridization probe moiety comprising a first PNA oligomer; and
   (b) a second conjugate comprising
      (i) a second hybridization probe moiety comprising a second PNA oligomer complementary to the first PNA oligomer; and
      (ii) a diagnostic agent or a therapeutic agent moiety;
   wherein the length of the second PNA oligomer in the second hybridization probe is not more than 14 bases.
2. The kit according to item 1 wherein the said target site resides on a mammalian, including human, protein which is expressed on the surface of a cell, preferably a tumor cell.
3. The kit according to item 2 wherein the said target site resides on a mammalian, including human, protein selected from the group consisting of epidermal growth factor receptor (EGFR), human epidermal growth factor receptor 2 (HER2), human epidermal growth factor receptor 3 (HER3), insulin-like growth factor 1 receptor (IGF1R), carbonic anhydrase IX (CAIX), platelet-derived growth factor receptor β (PDGFR-β), nectin-4, cluster of differentiation 38 (CD38), cluster of differentiation 33 (CD33), cluster of differentiation 30 (CD30), cluster of differentiation 22 (CD22), and cluster of differentiation 79b (CD79b).
4. The kit according to item 3 wherein the said target site resides on human epidermal growth factor receptor 2 (HER2).
5. The kit according to any one of items 1 to 4 wherein the said targeting moiety is selected from the group consisting of:
   antibodies;
   antibody fragments such as single-domain antibodies (sdAb; nanobodies), single-chain variable fragments (scFv), antigen-binding fragments (Fab), diabodies, or minibodies;
   engineered scaffold proteins such as Affibody® molecules; and
   peptides.
6. The kit according to item 5 wherein the said targeting moiety is an Affibody® molecule.
7. The kit according to any one of items 1 to 6 wherein one or both of the hybridization probes comprise a linker.
8. The kit according to item 7 wherein the said linker is a solubilizing moiety, preferably (a) a PEG-based linker, such as a linker comprising 2-[2-(2-aminoethoxy)ethoxy]acetic acid (AEEA);
   or (b) a peptide-based linker comprising charged or polar amino acids.
9. The kit according to any one of items 1 to 8 wherein the length of the first PNA oligomer in the first hybridization probe moiety is at least the length of the second PNA oligomer in the second hybridization probe moiety, and not more than 15 bases.
10. The kit according to item 9 wherein the length of said PNA oligomer in the first hybridization probe moiety is 15 bases.
11. The kit according to item 10 wherein the said 15 bases have the sequence c-c-t-g-g-t-g-t-t-g-a-t-g-a-t (SEQ ID NO: 3).
12. The kit according to item 11 wherein the said first hybridization probe moiety has the following structure:
   G-S-S-c-c-t-g-g-t-g-t-t-g-a-t-g-a-t-E-K(DOTA)-AEEA-E-NH₂.
13. The kit according to any one of items 1 to 12 wherein the length of said complementary PNA oligomer in the second hybridization probe moiety is between and 14 bases.
14. The kit according to item 13 wherein the length of said complementary PNA oligomer is between 9 and 12 bases.
15. The kit according to item 14 wherein the length of said complementary PNA oligomer is 9 bases.
16. The kit according to item 15 wherein the said 9 bases have the sequence a-a-c-a-c-c-a-g-g (SEQ ID NO: 4).
17. The kit according to item 16 wherein the said second hybridization probe moiety has the following structure:
   DOTA-AEEA-S-S-a-a-c-a-c-c-a-g-g-E-E-Y—NH₂.
18. The kit according to item 14 wherein the length of said complementary PNA oligomer is 12 bases.
19. The kit according to item 18 wherein the said 12 bases have the sequence a-t-c-a-a-c-a-c-c-a-g-g (SEQ ID NO: 5).
20. The kit according to item 19 wherein the said second hybridization probe moiety has the following structure:
   DOTA-AEEA-S-S-a-t-c-a-a-c-a-c-c-a-g-g-E-E-Y—NH₂.
21. The kit according to any one of items 1 to 20 wherein the said therapeutic agent is selected from the group consisting of radionuclides, calicheamicin, auristatins such as monomethyl auristatin E/F (MMAE/F), and maytansinoids such as maytansine derivatives DM0-DM4.
22. The kit according to item 21 wherein the said therapeutic agent is a radionuclide selected from the group consisting of Lutetium-177 (¹⁷⁷Lu), Yttrium-90 (⁹⁰Y), Bismuth-212 (²¹²Bi), Bismuth-213 (²¹³Bi), Astatine-211 (²¹¹At), Actinium-255 (²⁵⁵Ac), Copper-67 (⁶⁷Cu), Gallium-67 (⁶⁷Ga), and Rhenium-186 (¹⁸⁶Re).
23. The kit according to any one of items 1 to 22 wherein the said diagnostic agent generates a signal that is detectable by a method selected from the group consisting of positron emission tomography (PET), single photon emission computed tomography (SPECT), and optical imaging.
24. The kit according to item 23 wherein the said diagnostic agent is a radionuclide.
25. The kit according to item 24 wherein the method is SPECT and the radionuclide is selected from the group consisting of Technetium-99m (^{99m}Tc), Indium-111 (¹¹¹In), Lutetium-177 (¹⁷⁷Lu), Iodine-123 (¹²³I), Iodine-125 (¹²⁵I), Gallium-67 (⁶⁷Ga), and Copper-67 (⁶⁷Cu).
26. The kit according to item 24 wherein the method is PET and the radionuclide is selected from the group consisting of Gallium-68 (⁶⁸Ga), Fluorine-18 (¹⁸F), Iodine-122 (¹²²I), Iodine-124 (¹²⁴I), and Copper-64 (⁶⁴Cu).
27. The kit according to item 21 or 24 wherein the diagnostic agent or therapeutic agent is a radionuclide, and at least the second hybridization probe moiety comprises a chelator.
28. The kit according to item 27 wherein the said chelator is selected from the group consisting of:
1,4,7,10-tetraazacyclododecane-1,4,7,10-tetraacetic acid (DOTA),
1,4,7-triazacyclononane-1,4,7-triacetic acid (NOTA),
1,4,8,11-tetraazacyclododecane-1,4,8,11-tetraacetic acid (TETA),
1,4,7-triazacyclononane, 1-glutaric acid-4,7-acetic acid (NODAGA), and diethylenetriaminepentaacetic acid (DTPA).
   29. The kit according to item 28 wherein the said chelator is 1,4,7,10-tetraazacyclododecane-1,4,7,10-tetraacetic acid (DOTA).
   30. A method for delivering a diagnostic or therapeutic agent to a target site in a mammal, including humans, said method comprising:
      (a) administering to said mammal a first conjugate comprising
         (i) a targeting moiety which binds selectively to the target site; and
         (ii) a first hybridization probe moiety comprising a first PNA oligomer;
      (b) optionally, administering to said mammal a clearing agent, and allowing said clearing agent to clear non-localized first conjugate from circulation; and
      (c) administering to said mammal a second conjugate comprising
         (i) a second hybridization probe moiety comprising a second PNA oligomer complementary to the first PNA oligomer; and
         (ii) a diagnostic agent or a therapeutic agent moiety;
      wherein the length of the second PNA oligomer in the second hybridization probe moiety is not more than 14 bases, and wherein said second PNA oligomer binds to the first PNA oligomer in the first conjugate, thereby targeting the diagnostic agent or therapeutic agent moiety to the target site.
   31. A method for the diagnosis, prognosis or treatment of a medical condition in a mammal, including humans, said method comprising the method according to item for delivering a diagnostic or therapeutic agent moiety to a target site in the mammal.
   32. A method for the diagnosis of a medical condition, said method comprising administering to a mammal, including humans, the first and second conjugates of a kit according to any one of items 1 to 29.
   33. A method for the treatment of a medical condition, said method comprising administering to a mammal, including humans, in need of such treatment, the first and second conjugates of a kit according to any one of items 1 to 29.
   34. The method according to any one of items 31 to 33, wherein said medical condition is selected from the group consisting of cancer, infectious diseases, inflammatory diseases, and autoimmune diseases; preferably cancer.
   35. The method according to item 34, wherein said medical condition is cancer capable of forming solid tumors, said cancer selected from the group consisting of breast cancer, prostate cancer, lung cancer, head- and neck cancer, gastric cancer, and colon cancer.
   36. The method according to item 35, wherein said target site resides on a human protein selected from the group consisting of epidermal growth factor receptor (EGFR), human epidermal growth factor receptor 2 (HER2), human epidermal growth factor receptor 3 (HER3), insulin-like growth factor 1 receptor (IGF1R), carbonic anhydrase IX (CAIX), platelet-derived growth factor receptor β (PDGFR-β), and nectin-4.
   37. The method according to item 34, wherein said medical condition is hematological cancer selected from the group consisting of melanoma, leukemia, and myeloma.
   38. The method according to item 37, wherein said target site resides on a human protein selected from the group consisting of: cluster of differentiation 38 (CD38), cluster of differentiation 33 (CD33), cluster of differentiation 30 (CD30), cluster of differentiation 22 (CD22), and cluster of differentiation 79b (CD79b).
   39. A pharmaceutical composition comprising the first and second conjugates of a kit according to any one of items 1 to 29.
   40. The pharmaceutical composition according to item 39, for use in the diagnosis or treatment of a medical condition selected from the group consisting of cancer, infectious diseases, inflammatory diseases, and autoimmune diseases.

### EXPERIMENTAL METHODS

High quality water was used to prepare all buffer solutions and purified from metal contamination using Chelex® 100 resin (Bio-Rad Laboratories, USA). Carrier-free ¹⁷⁷LuCl₃ was purchased from PerkinElmer (Waltham, MA, U.S.A.). Radioactivity was measured using an automated gamma-spectrometer with a NaI(TI) detector (1480 Wizard, Wallac, Finland).

In vitro cell studies were performed using the HER2-expressing ovarian cancer SKOV3 and breast cancer BT474 cells, both obtained from the American Type Culture Collection (ATCC). Cells were cultured in RPMI medium (Flow Irvine, UK) supplemented with 10% fetal calf serum, 2 mM L-glutamine, and PEST composed of 100 IU/mL penicillin and 100 mg/mL streptomycin.

Data on in vitro studies and biodistribution were analyzed by unpaired 2-tailed t test (for comparison of two sets of data) and ANOVA (for comparison of several sets of data) using GraphPad Prism (version 4.00 for Windows; GraphPad Software) to determine significant differences.

Synthesis and Purification of PNA Pretargeting Probes

Peptide nucleic acid monomers; Fmoc-PNA-A(Bhoc)-OH, Fmoc-PNA-G(Bhoc)-OH, Fmoc-PNA-C(Bhoc)-OH and Fmoc-PNA-T-OH, were purchased from PolyOrg, Inc.

Leominster, USA or from PNA Bio, Inc. Thousand Oaks, USA. Rink Amide resin (ChemMatrix, 0.50 mmol/g) was purchased from Biotage (Uppsala, Sweden). 1,4,7,10-Tetraazacyclododecane-1,4,7,10-tetraacetic acid (DOTA) was purchased from CheMatech, Dijon, France. Fmoc-NH-(PEG)₂-CH₂COOH (AEEA) was purchased from Merck KGaA, Darmstadt, Germany. Solvents and reagents for solid-phase synthesis were obtained from commercial suppliers and used without further purification.

HP15 was synthesized on a Biotage Initiator+Alstra microwave peptide synthesizer using Rink Amide resin (ChemMatrix, 0.50 mmol/g) on a 50 μmol scale in a 10 ml reactor vial. Fmoc deprotection was performed at RT in two stages by treating the resin with piperidine-DMF (1:4) for 3 min followed by piperidine-DMF (1:4) for 10 min. Couplings were performed using 5 eq of PNA or amino acid monomer, 5 eq Oxyma and eq DIC in DMF. A coupling time of 10 min at 75° C. was used throughout the sequence followed by a capping step using NMP-lutidine-acetic anhydride (89:6:5) for 2 min.

Orthogonally protected Lys(Mtt) was introduced for the possibility of site-specific introduction of DOTA. Automated synthesis was interrupted after four coupling steps (Fmoc-E-K(Mtt)-[AEEA]-E-resin) for selective side chain deprotection of Lys(Mtt) with 5-10 additions of fresh TFA:TIS:DCM (1:2:97) followed by 1 min vortexing. Coupling of DOTA was performed using 5 eq. of DOTA, 5 eq Oxyma and 5 eq DIC in DMF at RT for 1 h. After resuming the automated synthesis and completion of all cycles, the resin was washed with DMF, DCM and finally with MeOH and then dried overnight. The PNA-peptide hybrid was cleaved from the solid support using a mixture of TFA:H₂O:TIS (95:2.5:2.5) for 4 h at RT. The PNA product was finally extracted between diethyl ether and water and lyophilized from the aqueous phase.

The shortest complementary PNA probe, HP19, was synthesized on the same microwave peptide synthesizer as HP15. Fmoc deprotection, couplings and capping were performed analogously to the synthesis of HP15. DOTA chelator was manually coupled to the PNA-probe at the end of the synthesis, using same protocol as DOTA coupled to HP15. The final product was cleaved from resin and extracted analogously to HP15. The synthesis was continuously monitored by Kaiser test. The molecular weight of the final product of HP19 was verified using MALDI-TOF analysis.

The other complementary PNA probes (HP16, HP17, HP18) were synthesized manually using same monomers, resin and solvents as used for the synthesis of HP15. Each coupling was performed using 5 eq PNA monomers. PNA monomers were pre-activated for 1 min with 5 eq benzotriazol-1-yl-oxytripyrrolidinophosphonium hexafluorophosphate (PyBOP; Sigma Aldrich) in the presence of 10 eq DIEA in NMP and DMF before addition to the resin. Each coupling was performed for 30 min to 1 h at RT with gentle shaking. Capping of unreacted PNA was performed analogously to HP15. Fmoc deprotection was performed using 20% piperidine in NMP for 20 min at RT with gentle shaking. The synthesis was monitored by Kaiser tests, and micro-cleavage of a few beads of resin followed by MALDI-TOF analysis. After completed synthesis, the resin was thoroughly washed with NMP followed by DCM and dried overnight. The subsequent cleavage and ether extraction of the complementary PNA probes were conducted analogously to HP15.

RP-HPLC purification was performed using a semi-preparative Zorbax 300 SB-C18 column (9.4×250 mm, 5 μm particle size; Agilent, Santa Clara, USA) with a linear gradient of 5-50% B, where A=0.1% TFA-H₂O and B=0.1% TFA-CH₃CN, over 25 min, using a flow rate of 3 ml/min, a column temperature of 70° C. and UV detection at 220 and 260 nm. Collected fractions were analyzed by MALDI-TOF (4800 MALDI-TOF/TOF, AB SCIEX) using α-cyano-4-hydroxycinnamic acid matrix. Fractions determined to contain the correct products were pooled and lyophilized.

The purity of HP16, HP17 and HP18 was confirmed using analytical RP-HPLC on a Zorbax 300SB-C18 column (4.6×150 mm, 3.5 μm particle size; Agilent) followed by MALDI-TOF analysis. The identity and purity of HP19 was confirmed using MALDI-TOF analysis. Extinction coefficients at 260 nm (6260) were estimated for each PNA probe based on the PNA composition and the extinction coefficient of each PNA monomer (A: 13 700 M⁻¹cm⁻¹, C: 6 600 M⁻¹cm⁻¹, G: 11 700 M⁻¹cm⁻¹, and T: 8 600 M⁻¹cm⁻¹). Extinction coefficients used throughout all experiments for each probe are the following; HP16: 98 000 M⁻¹cm⁻¹, HP17: 126 900 M⁻¹cm⁻¹, HP18: 155 800 M⁻¹cm⁻¹, HP19: 64 000 M⁻¹cm⁻¹ and HP15: 150 700 M⁻¹cm⁻¹.

Production of Affibody-PNA Conjugate

The pAY430-Z_{HER2:342}-SR-H6 plasmid (Westerlund 2015) was transformed into BL21 (DE3) chemically competent E. coli cells (Life Technologies), and the cells were cultivated in complex media (tryptic soy broth with yeast extract) supplemented with kanamycin. Protein expression was induced by the addition of 1 mM IPTG (final concentration), and the culture was kept on 150 rpm at RT overnight. Cells were harvested by centrifugation (4000 rcf, 10 min, 4° C.), resuspended in IMAC binding buffer (20 mM Tris-HCl, 300 mM NaCl, 10 mM imidazole, pH 7.5) and subsequently lysed by sonication. After centrifugation, the clarified supernatant was passed through IMAC matrix (HisPur™ Cobalt Resin, Thermo Scientific) capturing Z_{HER2:342}-SR-H6. The resin was washed with wash buffer (20 mM Tris-HCl, 300 mM NaCl, 30 mM imidazole, pH 7.5), and the protein eluted using elution buffer (20 mM Tris-HCl, 300 mM NaCl, 300 mM imidazole, pH 7.5). Eluted Z_{HER2:342}-H6 was buffer-exchanged to sortase A conjugation buffer (50 mM Tris-HCl, 150 mM NaCl, 10 mM CaCl₂ pH 7.5) on a PD-10 desalting column (GE Healthcare). The purity and molecular weight of Z_{HER2:342}-SR-H6 was confirmed using SDS-PAGE and MALDI-TOF.

Z_{HER2:342}-SR-H6 was site-specifically conjugated to HP15 using sortase A-mediated ligation (SML). The SML method described below is based on a protocol for affibody-PNA conjugation previously published (Altai 2017). A sortase A variant with P94S/D160N/K196T (Chen et al. 2011) mutations, denoted Srt A3*, was utilized for the conjugation. The glycine-modified HP15 probe was dissolved in 10% DMSO and heated at 80° C. for 5 min before the concentration was estimated based on the absorbance at 260 nm. 500 nmol HP15, 1.25 μmol of Z_{HER2:342}-SR-H6 and 1.25 μmol NiCl₂ were mixed in sortase A conjugation buffer at a final volume of 5 ml. Srt A3* was added to the reaction to a final concentration of 5 μM and the reaction proceeded for 30 min and was subsequently subjected to a reverse IMAC step using pre-equilibrated IMAC matrix. The conjugation product, hydrolyzed protein by-products and unconjugated HP15 could be collected in the flow-through after a 30 min incubation step with the given matrix. Collected flow-through was then subjected to buffer exchange to 10 mM NaOAc pH 3.6 on PD-10 column and subsequently lyophilized. Z_{HER2:342}-SR-HP15 conjugate was purified on RP-HPLC using the same column and solvents as for purification of PNA probes but with a gradient going from 5% to 50% B in 25 min, and the absorbance was monitored at 220, 260 and 280 nm. Unconjugated HP15 and Z_{HER2:342}-SR-HP15 conjugate fractions were collected, lyophilized and analyzed by MALDI-TOF. To confirm the purity of the purified conjugate it was analyzed by analytical HPLC (Zorbax 300SB-C18, 3.5 μm particle size, 4.6×150 mm, Agilent) and electrospray ionization mass spectrometry (ESI-MS, Impact II, Bruker). Unconjugated HP15 could be used for new conjugation reactions together with a new batch of Z_{HER2:342}-SR-H6.

Final concentrations of the PNA probes and Z_{HER2:342}-SR-HP15 were determined by measuring absorbance at 260 nm. The same extinction coefficient was used for both HP15 and Z_{HER2:342}-SR-HP15 as the contribution from the protein part to the total absorbance at 260 nm would be negligible.

Binding of Z_{HER2:342}-SR-HP15 to the HER2 receptor was confirmed by surface plasmon resonance (SPR).

Characterization of the PNA Pretargeting Probes

The kinetic parameters for hybridization of the PNA probes were analyzed using surface plasmon resonance (SPR) on a Biacore™ 8K instrument (GE Healthcare). Dextran chips Series S Sensor CM5 (GE Healthcare), were functionalized with Z_{HER2:342}-SR-HP15 on four surfaces to 385, 194, 185 and 353 resonance units (RU) using standard amine coupling procedures. A reference surface on each chip was subjected to activation followed by deactivation. The complementary PNA probes HP16, HP17, HP18 and HP19 were injected at 5 concentrations; 22.6, 45.3, 90.6, 181.25 and 362.5 nM using a single cycle injection. Association for each concentration was allowed for 300 s, followed by the next injection and association phase. After injection of the final concentration, dissociation was allowed for 10 000 s (2 h 47 min) before regeneration with 10 mM HCl for 30 s followed by 15 mM NaOH for 30 s. All runs were performed in PBST (0.05% Tween-20) pH 7.4 using a flow rate of 50 11.1/min at 25° C. Kinetic parameters were calculated using 1:1 binding model in Biacore Insight Evaluation software.

Melting temperatures for HP15:HP16, HP15:HP17 and HP15:HP18 were determined by monitoring UV absorbance at 260 nm (Chirascan™, Applied Photophysics) at a temperature range from 20 to 95° C., using a temperature change of 1° C./min. The PNA complexes were heated to 80° C. for 5 min and were then allowed to hybridize at room temperature for 5 min prior to UV monitoring at room temperature for 5 min. CD spectra were collected before and after determination of thermal denaturation. The melting temperature for the HP15:HP19 complex was determined using a Varian Cary 50 Bio UV-visual spectrophotometer equipped with a single-cell Peltier thermostat controlled cuvette holder. The temperature in the cell was adjusted at a speed of 0.5° C./min between 20 to 80° C., and measurements were taken at 260 nm after a 60 s equilibration at each temperature point.

Radiolabeling and In Vitro Stability

Radiolabeling of the primary and secondary PNA probes with ¹⁷⁷Lu was performed using a previously described method (Westerlund 2018). Briefly, 30 μg of peptide was dissolved in 100 μL of ascorbic acid (1 M, pH 5.5) by heating at 95° C. for 10 min followed by sonication for 5 min to ensure total dissolving. 3 μL (60-120 MBq) of ¹⁷⁷LuCl₃ was added followed by vortexing. The mixture was incubated at 95° C. for 60 min. The reaction mixture was analyzed by radio-ITLC, eluted with 0.2 M citric acid, pH 2.0.

To remove loosely bound ¹⁷⁷Lu, a treatment with excess of ethylenediaminetetraacetic acid tetra sodium salt (EDTA·Na₄) was performed. A freshly prepared solution of EDTA·Na₄ (10 mg/mL in Milli-Q water), was added in 1000-fold molar excess to the reaction mixture and incubated at 95° C. for 10 min. No further purification was needed for new secondary probes due to the high radiochemical yield and purity. For [¹⁷⁷Lu]Lu-HP2 and [¹⁷⁷Lu]Lu-Z_{HER2:342}-SR-HP15, a purification was performed after EDTA treatment, by size exclusion chromatography using disposable NAP-5 columns, pre-equilibrated and eluted with 1% BSA/PBS.

To evaluate stability, a fraction of the freshly radiolabeled conjugate (0.4 μg) was incubated with 500-fold molar excess of EDTA for 60 min at 37° C. Incubation was also performed in PBS as a control. The test was run in triplicates.

To validate the results of radio-ITLC, reversed phase-HPLC conducted on an LaChrom Elite® system (Hitachi, VWR, Darmstadt, Germany) consisting of an L-2130 pump, a UV detector (L-2400), and a radiation flow detector (Bioscan, Washington, DC, USA) coupled in series was used. Purity analysis of [¹⁷⁷Lu]Lu-labeled compounds was performed using an analytical column (Phenemenex, Aschaffenburg, Germany; Luna® 5 μm C18, 100 Å; 150×4.6 mm column). The HPLC conditions were as follows: A=10 mM TFA/H₂O; B=10 mM TFA/acetonitrile; UV-detection at 220 nm; gradient elution: 0-15 min at 5 to 70% B, 15-18 min at 70 to 95% B, 19-20 min at 5% B; and flow rate was 1.0 mL/min.

In Vitro Studies

Cells were seeded in cell-culture dishes with a density of 10 6 cells/dish. A set of three dishes was used for each data point.

The specificity of [¹⁷⁷Lu]Lu-Z_{HER2:342}-SR-HP15 binding to HER2-expressing cells was tested by incubation of cells with 1 nM of labeled conjugate for 1 h at 37° C. To saturate receptors, unlabeled Z_{HER2:342} (1000 nM) was added to a control set 5 min before adding radiolabeled probe.

Pretargeting specificity assay for novel [¹⁷⁷Lu]Lu-HP16, [¹⁷⁷Lu]Lu-HP17 and [¹⁷⁷Lu]Lu-HP18 was performed using four sets of cell dishes as described earlier (Honarvar 2016). To demonstrate the pretargeting, one set of dishes was incubated with Z_{HER2:342}-SR-HP15 (1 nM) for 1 h at 37° C. and washed. A ¹⁷⁷Lu-labeled secondary probe (10 nM) was added and cells were incubated for 1 h at 37° C. To show that the pretargeting was HER2-mediated, the second set of dishes was incubated with an excess of affibody molecules Z_{HER2:342} (1000 nM) for 5 min before adding Z_{HER2:342}-SR-HP15. A ¹⁷⁷Lu-labeled secondary probe (10 nM) was added and cells were incubated for 1 h at 37° C. To demonstrate that pretargeting was PNA-mediated, the third set of dishes was incubated with Z_{HER2:342}-SR-HP15 followed by incubation with an excess of non-labeled secondary probe (300 nM) for 30 min and then the ¹⁷⁷Lu-labeled secondary probe was added followed by 1 h incubation. In the fourth set, the cells were incubated only with ¹⁷⁷Lu-labeled secondary probe to assess non-specific binding. At the end of incubation, the cells were washed and detached by trypsin, the radioactivity in cells was measured to calculate the percent of cell-bound radioactivity.

To evaluate the binding affinity of the radiolabeled conjugates to HER2 receptors, the kinetics of binding of [¹⁷⁷Lu]Lu-labeled probes to and their dissociation from SKOV3 cells were measured using a LigandTracer® yellow instrument (Ridgeview Instruments AB, Vänge, Sweden). SKOV3 cells were seeded on a local area of a cell culture dish (Nunclon™, Size 100620, NUNC A/S, Roskilde, Denmark). SKOV3 cells were pre-saturated with Z_{HER2:342}-SR-HP15 (1 nM) in two set of dishes for 2 h and thereafter, washed three times to remove unbound primary agent. Two increasing concentrations of the radiolabeled molecules (for [¹⁷⁷Lu]Lu-Z_{HER2:342}-SR-HP:15: 180 and 540 pM, and for [¹⁷⁷Lu]Lu-labeled secondary probes:1 and 5 nM) were added. The data was analyzed using the Interaction Map™ software (Ridgeview Diagnostics AB, Uppsala, Sweden) to calculate association rate constant (kₐ), dissociation rate constant (k_{d}) and equilibrium dissociation constant (K_{D}). Analysis was performed in duplicates.

Cellular processing and retention on SKOV3 and BT474 cells were studied during interrupted incubation by an acid-wash method (Wållberg 2008).

In Vivo Studies

Animal experiments were performed in accordance with the national legislation for work with laboratory animals. Approval was granted by the Ethical Committee for Animal Research in Uppsala. For tumor implantation, 10⁷ SKOV3 cells were subcutaneously injected on the right hind leg of female BALB/c nu/nu mice. The biodistribution experiments were performed two weeks after cell implantation. The average animal weight was 18±1 g. The average tumor weight was 0.23±0.11 g. For biodistribution measurement, the mice were euthanized at predetermined time points by overdosing of anesthesia (Rompun®/Ketalar®) followed by heart puncture. The organs of interest and the tumor were collected and weighed, and their radioactivity was measured. The percentage of the total injected dose per gram of sample (% ID/g) was calculated.

The pretargeting protocol used in this study has been previously optimized for the affibody-based PNA-mediated therapy using [¹⁷⁷Lu]Lu-HP2 by Westerlund et al. (Westerlund 2018). Upscaling experiments showed no significant changes in the biodistribution of [¹⁷⁷Lu]Lu-HP2, when the injected mass of both primary and [¹⁷⁷Lu]Lu-HP2 was doubled. Based on this study, 50 μg of primary agents and equimolar amounts of secondary agents (0.69, 0.89, 1 and 1 μg for HP16, HP17, HP18 and HP2, respectively) were injected per mouse.

For biodistribution studies, mice were randomized into groups of five. The 30 mice were intravenously injected with Z_{HER2:342}-SR-HP15 (4 nmol in 100 μL PBS per mouse). Sixteen hours later, all mice were injected with [¹⁷⁷Lu]-HP16, [¹⁷⁷Lu]-HP17 or [¹⁷⁷Lu]-HP18 (194 pmol in 100 μL 2% BSA in PBS, 120 kBq). The biodistribution was measured at 4 and 144 h after injection of secondary probes. For comparison, biodistribution of first generation [¹⁷⁷Lu]Lu-HP2 was measured in the same way using Z_{HER2:342}-SR-HP:1 as primary agent.

To evaluate in vivo specificity, the biodistribution of [¹⁷⁷Lu]Lu-secondary probes was measured 4 h after injection without pre-injection of primary agent.

After the gamma-counter measurements were completed, tumors were embedded in a cryomedium (Neg50™, Thermo Scientific, USA) and frozen at −80° C. Frozen tumors were cut in serial sections (30 μm thick) using a cryomicrotome (CryoStar™ NX70, Thermo Scientific, USA) and thaw-mounted on glass slides. For the digital autoradiography, the slides with sections were put in a cassette and exposed to phosphor screens overnight. The phosphor screens were scanned on a Cyclone® Storage Phosphor System at 600 dpi resolution and analyzed using the OptiQuant software (PerkinElmer, USA).

To estimate a ratio of absorbed doses in tumor and kidneys, cumulated activity in kidney and tumor were assessed. The estimation was based on a clinically validated two time point approach (Freedman 2020). The biodistribution data were non-decay corrected and areas under time-activity plot was calculated using GraphPad Prism software. The assumptions were that the main absorbed dose will be due to beta-particles, as cross-doses would be negligible, and the absorbed fraction would be equal to 1.

SPECT/CT Imaging

Mice bearing SKOV3 xenografts were injected intravenously with 7 nmol of primary agent 16 h before injection of secondary ¹⁷⁷Lu-labeled probes (680 pmol, 9-13 MBq). Immediately before imaging (4 h after injection), the animals were sacrificed by CO₂ asphyxiation. SPECT imaging was performed using nanoScan SC (Mediso Medical Imaging Systems, Hungary). CT acquisitions were carried out using X-ray energy of 50 keV. 20-min SPECT helical scans were acquired using energy windows 50-62, 103-124, and 188-230 keV. The data were reconstructed using Tera-Tomo™ 3D SPECT Software.

### EXAMPLES OF THE INVENTION

### Example 1: Production and Characterization of the Affibody-PNA Conjugate and the Complementary PNA Probes

The PNA pretargeting probes HP15, HP16, HP17, HP18 and HP19 (Table 1) were prepared as described above in Experimental Methods. The probes were designed to avoid self-complementary sequences as well as extended stretches of purines (A and G), which are known to promote aggregation and can thus make the PNA-based probes difficult to synthesize and purify (Zhao 2020).

The Affibody® molecule Z_{HER2:342}-SR-H6 and HP15 were conjugated using Srt A3* (see Experimental Methods). The ligation efficiency for the reaction was estimated to 40% based on integrated areas under peaks at 260 nm in RP-HPLC. The ligation efficiency of 40% is lower than the previously reported ligation efficiency of 70% (Altai 2017) for conjugation of HP1 to Z_{HER2:342}-SR-H6. However, HP15 has a single glycine residue at the N-terminus, compared to three glycine residues at the N-terminus of HP1, which might influence the ligation efficiency. The ligation efficiency of 40% is in the same range as conjugation of HP1 using wild type sortase A, which was reported to 45% (Westerlund, 2015).

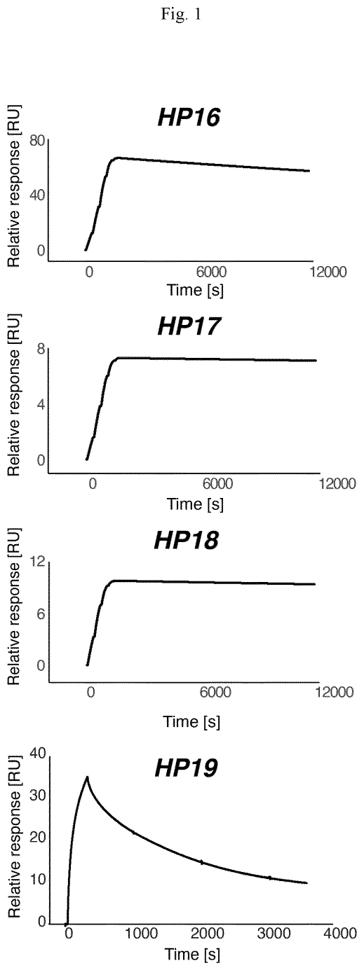

Hybridization of the four complementary PNA probes to immobilized Z_{HER2:342}-SR-HP15 was analyzed by SPR. Representative sensorgrams of the interactions, analyzed using single-cycle injection, are shown in FIG. 1. The association rate constants, kₐ, were estimated to 4.6×10⁴, 4.3×10⁴, and 5.7×10⁴ M⁻¹s⁻¹ for HP16, HP17 and HP18, respectively. Hence, the on-rates for all of these three PNA probes interacting with HP15 are within the same range. The k a was estimated to 2.1×10⁷ M⁻¹s⁻¹ for HP19. The dissociation rate constant, k_{d}, was estimated to 1.2×10⁻⁵ s⁻¹ for HP16 and 7.2×10⁻² s⁻¹ for HP19, whereas for the other two complementary PNA probes (HP17 and HP18), the k_{d} was too slow to be determined using Biacore. The equilibrium dissociation constant for HP16 was calculated to be approximately 280 pM (Table 2), while the affinity for HP17 and HP18 to Z_{HER2:342}-SR-HP15 was estimated to be higher.

The K_{D} (280 pM) determined for the interaction between HP15 and HP16 (9-mer), appears to indicate sufficiently high affinity for the intended pretargeting application. The K_{D} (3.4 nM) determined for the interaction between HP15 and HP19 (6-mer) is higher, but also this lower affinity is expected to be sufficient for the application. Successful pretargeting has earlier been demonstrated using bispecific antibody constructs, binding both a tumor-associated antigen and a radiolabeled hapten, with an estimated K_{D} for the interaction between the bifunctional antibody and an ¹¹¹In-labeled benzyl EDTA derivative of only 10⁻⁹-10⁻¹⁰ M (Stickney 1991).

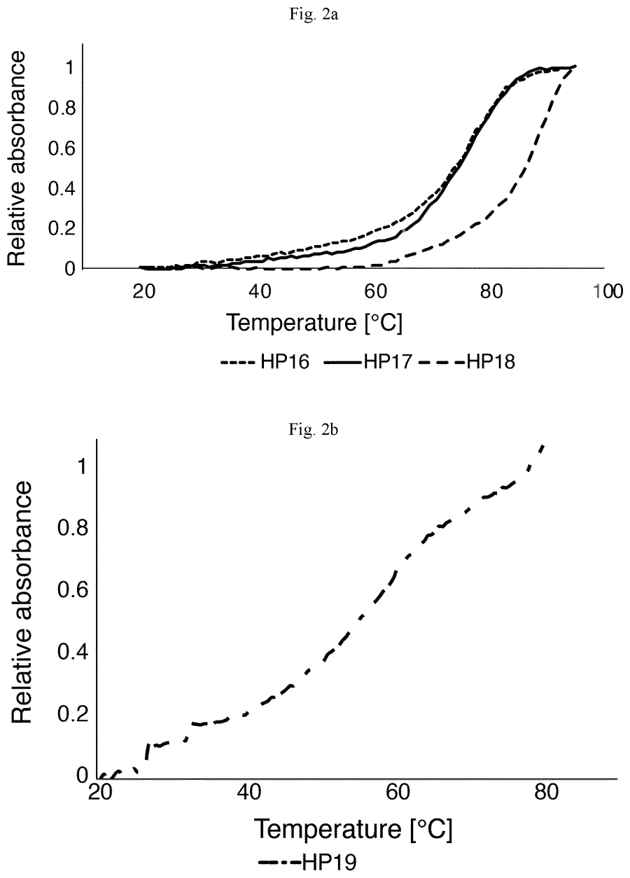

The hybridization between HP15 and the secondary probes H16, HP17 or HP18 gave rise to CD spectra with minima at approximately 215 nm and 260 nm. The induced signals are the result of PNA:PNA double helix formation upon hybridization between complementary PNA probes carrying C-terminal L-amino acids (Corradini 2012). Melting temperatures for each hybridization probe after duplex formation with HP15 were monitored at 260 nm and estimated to 73° C., 75° C. and 87° C. for HP16, HP17 and HP18, respectively (FIG. 2a) and to 55° C. for HP19 (FIG. 2b). The spectroscopic characterization illustrates that HP16, HP17 and HP18 all form duplexes of high thermal stability with the primary PNA probe HP15. The lower melting temperatures for HP16, HP17 and HP19 are expected because of their shorter oligomer lengths compared to HP18. While being significantly lower than those for HP16, HP17 and HP18, the melting temperature for HP19 is still well above human body temperature.

### Example 2: Radiolabeling and In Vitro Stability

Table 3 shows the results of radiolabeling of all probes with ¹⁷⁷Lu. The radiochemical yield for new secondary agents after EDTA treatment was over 98%. Therefore, no further purification using NAP-5 was performed for in vitro and in vivo studies. Purification of [¹⁷⁷Lu]Lu-Z_{HER2:342}-SR-HP15 and [¹⁷⁷Lu]Lu-HP2 using NAP-5 column resulted in 100±0% radiochemical purity for both labels. All probes labelled with ¹⁷⁷Lu were stable in PBS and in the presence of EDTA for up to 1 h incubation.

To validate the radio-ITLC results, radio-HPLC identity was performed and it demonstrated that no fragmentation occurred after labelling and purification. The radio-HPLC retention time of all probes was around 5.8 min. The retention time of the non-labelled probes was the same as labelled ones.

### Example 3: In Vitro Studies

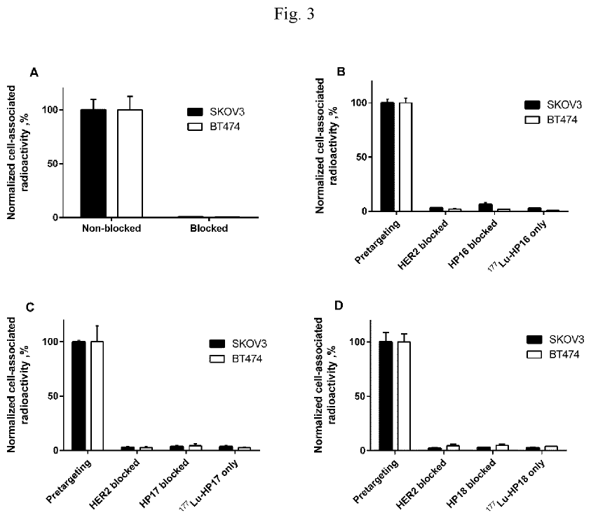

HER2-binding specificity of the primary agent [¹⁷⁷Lu]Lu-Z_{HER}2:342-SR-HP15 was tested using a saturation experiment. The binding was significantly (p<0.0005) decreased when the cells were pre-saturated with the anti-HER2 affibody molecule (FIG. 3A), demonstrating that the binding was HER2-mediated. The slow internalization of [¹⁷⁷Lu]Lu-Z_{HER2:342}-SR-HP15 is favorable for pretargeting application, as this enables long persistence of the primary hybridization probe on targeted cells' surface.

The specificity of [¹⁷⁷Lu]Lu-HP16, [¹⁷⁷Lu]Lu-HP17 and [¹⁷⁷Lu]Lu-HP18 in vitro binding to Z_{HER2:342}-SR-HP15-pretreated HER2-expressing cells was evaluated (FIGS. 3B, C, and D). The binding of all secondary agents was significantly (p<0.0005) decreased when HER2 receptors were saturated with anti-HER2 affibody molecule or when Z_{HER2:342}-SR-HP15-treated cells were pre-incubated with a large excess of non-labeled secondary agent. The binding of all secondary agents to cells without pre-incubation with the primary agent was significantly (p<0.0005) lower compared to pretargeting. The specificity of [¹⁷⁷Lu]Lu-HP2 has been demonstrated earlier (Altai 2017 NMB).

LigandTracer® measurements of kinetics of binding to living SKOV3 cells, which were pre-treated with Z_{HER2:342}-SR-HP15, demonstrated that the binding of all secondary probes was extremely strong. Interaction Map™ calculation showed a rapid association followed by very slow dissociation for [¹⁷⁷Lu]Lu-Z_{HER2:342}-SR-HP15 and pretargeted [¹⁷⁷Lu]Lu-secondary probes, resulting in picomolar dissociation constants at equilibrium (K_{D}). K_{D} values were between 11 and 12 pM. There was no difference in apparent dissociation constant between the secondary probes. Thus, reduction of length of PNA from 15 to 9 nitrogenous bases was not associated with any observable reduction of their binding to primary probe in the cell assay.

Cellular processing and retention of all radiolabels by SKOV3 and BT474 cells after interrupted incubation was determined. For [¹⁷⁷Lu]Lu-Z_{HER2:342}-SR-HP15, the internalization was slow, which is typical for HER2-binding affibody molecules and their derivatives. The internalized fraction of [¹⁷⁷Lu]Lu-Z_{HER2:342}-SR-HP15 was slightly higher in SKOV3 cells than BT474 cells (18±2% for SKOV3 vs. 12±2% for BT474 at 24 h time point). The pattern of internalization of labeled secondary probes was similar to the pattern of [¹⁷⁷Lu]Lu-Z_{HER2:342}-SR-HP15. Retention of radioactivity over time was highest for [¹⁷⁷Lu]Lu-Z_{HER2:342}-SR-HP15, while the [¹⁷⁷Lu]Lu-secondary probes showed faster release of bound radioactivity over time.

### Example 4: In Vivo Studies

The data concerning biodistribution of [¹⁷⁷Lu]Lu-labeled secondary probes without pre-injection of a primary probe are provided in Table 4. The biodistribution of [¹⁷⁷Lu]-HP16 and [¹⁷⁷Lu]-HP17 was very similar, except small but significant difference in bone uptake. In comparison with [¹⁷⁷Lu]-HP18, the shorter variants had significantly lower uptake in blood, liver and kidney. [¹⁷⁷Lu]-HP17 has also significantly lower uptake in lung and bone.

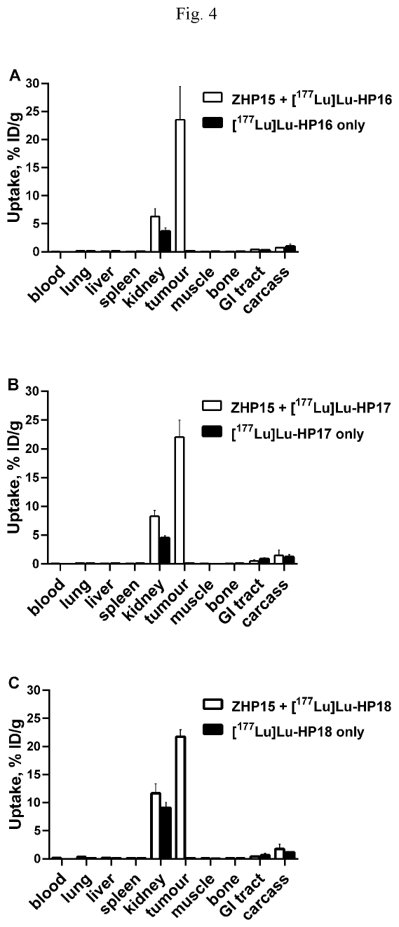

The results of the in vivo specificity of [¹⁷⁷Lu]Lu-secondary probes (4 h after injection) with and without the pre-injection of Z_{HER2:342}-SR-HP15 (4 nmol) are presented in FIG. 4. When mice were pre-injected with Z_{HER2:342}-SR-HP15, the tumor uptake of all [¹⁷⁷Lu]Lu-secondary probes was significantly (p<0.00005) higher than without pre-injection. Interestingly, uptake in normal tissues was also significantly higher in the case of pre-injection of primary probes.

The dramatic increase of uptake of the secondary probes in tumors after pre-injection of primary agent convincingly proves the specificity of pretargeting. Interestingly, an increased uptake of secondary probes after injection of Z_{HER2:342}-SR-HP15 was also observed in blood, kidneys, spleen, and muscles. This might be explained by association of the secondary probes with the primary agent, which had not completely cleared from circulation or re-entered the blood flow after dissociation from receptors in tumor. This effect was less pronounced for [¹⁷⁷Lu]-HP16.

The results from the comparative biodistribution of [¹⁷⁷Lu]Lu-HP16, [¹⁷⁷Lu]Lu-HP17, [¹⁷⁷Lu]Lu-HP18 with [¹⁷⁷Lu]Lu-HP2 after injections of primary probes in SKOV3-bearing mice are presented in Table 5.

The biodistribution measurements show a rapid clearance from blood and normal organs and tissues for all studied PNA-based probes. Some difference in biodistribution between conjugates were observed. The blood concentration was significantly (p<0.0001) higher for [¹⁷⁷Lu]Lu-HP18 than for the other secondary probes at 4 h after injection. At this time point, the hepatic uptakes for [¹⁷⁷Lu]Lu-HP16, [¹⁷⁷Lu]Lu-HP17 and [¹⁷⁷Lu]Lu-HP2 (0.1±0.0% ID/g) were equal but significantly (p<0.05) lower than for [¹⁷⁷Lu]Lu-HP18 (0.2±0.1% ID/g). The only tissues with prominent uptake were kidney and tumor. The uptake in kidney for [¹⁷⁷Lu]Lu-HP16 (6±1% ID/g) was significantly (p<0.05) lower than for [¹⁷⁷Lu]Lu-HP18 (12±2% ID/g) and [¹⁷⁷Lu]Lu-HP2 (10±2% ID/g). [¹⁷⁷Lu]Lu-HP16 showed the highest average tumor uptake (24±6% ID/g) but the difference to the uptakes of other probes was not significant. The combination of lower kidney uptake and high uptake in tumor resulted in higher tumor-to-kidney ratio for [¹⁷⁷Lu]Lu-HP16 (4-fold higher than renal uptake) at 4 h after injection. Renal uptake for [¹⁷⁷Lu]Lu-HP16 and [¹⁷⁷Lu]Lu-HP17 was significantly (p<0.005) lower than for [¹⁷⁷Lu]Lu-HP18. The order of tumor uptake was [¹⁷⁷Lu]Lu-HP17 and [¹⁷⁷Lu]Lu-HP18 (both 5±1% ID/g)>[¹⁷⁷Lu]Lu-HP2 (4±1% ID/g)>[¹⁷⁷Lu]Lu-HP16 (both 3±1% ID/g) at 144 h after injection. [¹⁷⁷Lu]Lu-HP17 showed better retention of radioactivity in tumor and faster clearance in kidney (tumor uptake was 7-fold higher than renal uptake), resulting in higher tumor-to-kidney ratio at 144 h after injection.

### Example 5: SPECT/CT Imaging

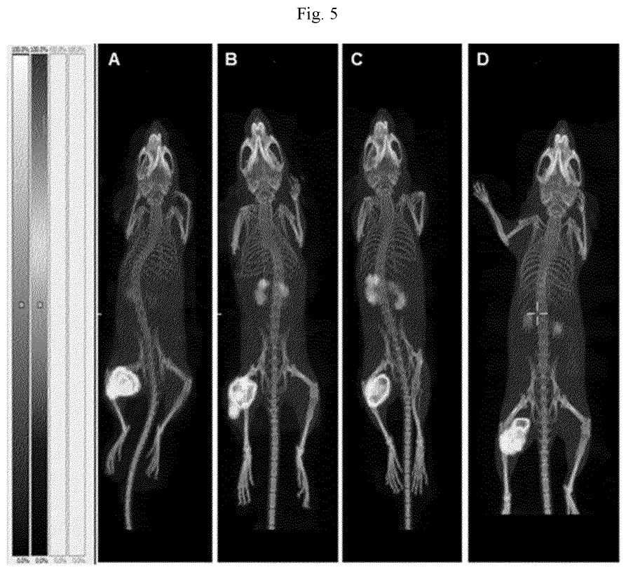

Results of SPECT/CT imaging (FIG. 5) confirmed efficient affibody molecule-based PNA-mediated pretargeting for all variants. The tumors were the sites with the highest uptake. The only tissues with noticeable uptake were kidneys and tumor. The radioactivity uptake in tumor was considerably higher than in kidneys in each animal.

Distribution of radioactivity in tumor was evaluated using autoradiography. The distribution of activity 4 h after injection was quite uniform and reflected cluster character of xenografts. At a later time point, a gradient of radioactivity concentration from tumors core to rims was more pronounced.

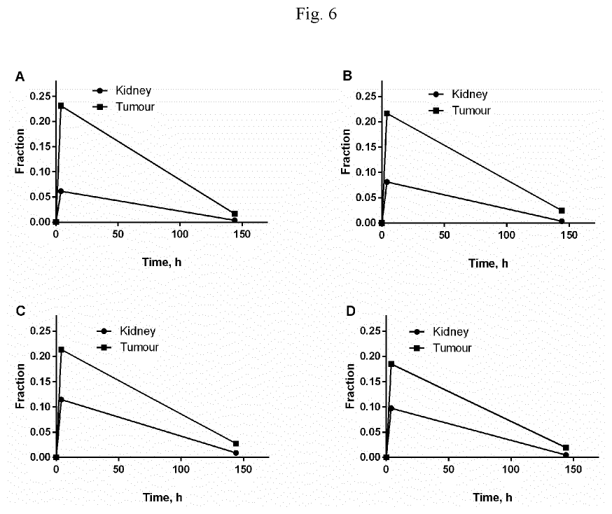

The results of the estimated dosimetry to kidney and tumor are shown in Table 6 and FIG. 6. The ratio of areas under time-activity plots for tumor and kidneys was 3.8, 2.8, 2.0 and 2.0 for HP16, HP17, HP18 and HP2, respectively. This indicates that [¹⁷⁷Lu]Lu-HP16 would provide the most favorable dosimetry for a therapy application.

### REFERENCES

Altai M, Varasteh Z, Andersson K, Eek A, Boerman O, Orlova A. In vivo and in vitro studies on renal uptake of radiolabeled affibody molecules for imaging of HER2 expression in tumors. Cancer Biother Radiopharm. 2013; 28:187-195.
Altai M, Perols A, Tsourma M, et al. Feasibility of affibody-based bioorthogonal chemistry-mediated radionuclide pretargeting. J Nucl Med. 2016; 57: 431-436.
Altai M, Westerlund K, Velletta J, Mitran B, Honarvar H, Karlström A E. Evaluation of affibody molecule-based PNA-mediated radionuclide pretargeting: Development of an optimized conjugation protocol and 177Lu labeling. Nucl Med Biol. 2017 November; 54:1-9.
Altai M, Membreno R, Cook B, Tolmachev V, Zeglis B M. Pretargeted Imaging and Therapy. J Nucl Med. 2017; 58:1553-1559.
Chen I, Dorr B M, Liu D R. A general strategy for the evolution of bond-forming enzymes using yeast display. Proc Natl Acad Sci USA 2011; 108:11399-404.
Corradini, R., Tedeschi, T., Sforza, S., and Marchelli, R. (2012) Electronic circular dichroism of peptide nucleic acids and their analogues. In Comprehensive Chiroptical Spectroscopy: Applications in Stereochemical Analysis of Synthetic Compounds, Natural Products, and Biomolecules (Berova, N., Polavarapu, P. L., Nakanishi, K., and Woody, R. W., Eds.),
Demidov V V, Potaman, V N, Frank-Kamenetskii M D, et al. Stability of peptide nucleic acids in human serum and cellular extracts. Biochem Pharmacol. 1994; 48: 1310-1313.
Egholm M, Buchardt O, Christensen L, et al. PNA hybridizes to complementary oligonucleotides obeying the Watson-Crick hydrogen-bonding rules. Nature. 1993; 365: 566-568.
Fortin M A, Orlova A, Malmstrom P U, Tolmachev V. Labelling chemistry and characterization of [⁹⁰Y/¹⁷⁷Lu]-DOTA-Z_{HER2:342}-3 Affibody molecule, a candidate agent for locoregional treatment of urinary bladder carcinoma. Int J Mol Med. 2007; 19: 285-291.
Freedman N, Sandström M, Kuten J, Shtraus N, Ospovat I, Schlocker A, Even-Sapir E. Personalized radiation dosimetry for PRRT-how many scans are really required? EJNMMI Phys. 2020 May 11; 7(1):26.
Frampas E, Rousseau C, Bodet-Milin C, Barbet J, Chatal J. F, and Kraeber-Bodere F. Improvement of radioimmuno-therapy using pretargeting. Front Oncol. 2013; 3: 159.
Garcia-Alonso S, Ocaña A, Pandiella A. Trastuzumab Emtansine: Mechanisms of Action and Resistance, Clinical Progress, and Beyond. Trends Cancer. 2020 February; 6(2):130-146.
Garousi J, Vorobyeva A, Altai A. Influence of several compounds and drugs on the renal uptake of radiolabeled affibody molecules. Molecules, 2020; 25(11):E2673.
Holliger, P., Prospero, T., & Winter, G. “Diabodies”: small bivalent and bispecific antibody fragments. Proc Natl Acad Sci USA 1993; 90(14), 6444-6448.
Honarvar H, Westerlund K, Altai M, et al. Feasibility of affibody molecule-based PNA-mediated radionuclide pretargeting of malignant tumors. Theranostics. 2016; 6: 93-103.
Hu, S et al. Minibody: A Novel Engineered Anti-Carcinoembryonic Antigen Antibody Fragment (Single-Chain Fv-CH3) Which Exhibits Rapid, High-Level Targeting of Xenografts. Cancer Res. 1996; 56:3055-3061.
Kreutzfeldt J, Rozeboom B, Dey N, De P. The trastuzumab era: current and upcoming targeted HER2+ breast cancer therapies. Am J Cancer Res. 2020 Apr. 1; 10(4):1045-1067.
Larson S M, Carrasquillo J A, Cheung N K, Press O W. Radioimmunotherapy of human tumours. Nat Rev Cancer. 2015 June; 15(6):347-60.
Liu R, Li X, Xiao W, Kit S, Lam KS. Tumor-targeting peptides from combinatorial libraries. Advanced Drug Delivery Reviews, 2017; 110-111:13-37.
Mather S J. 99Tc peptide-peptide nucleic acid probes for imaging oncogene mRNAs in tumours. Nucl Med Commun. 2004 April; 25(4):399.
Moody T W, Ramos-Alvarez I, Jensen R T. Neuropeptide G Protein-Coupled Receptors as Oncotargets. Front Endocrinol (Lausanne). 2018 Jun. 29; 9:345.
Nielsen P E, Egholm M, Buchardt, O. Peptide nucleic acid (PNA). A DNA mimic with a peptide backbone. Bioconjugate Chem. 1994; 5: 3-7.
Orlova A et al. Tumor Imaging Using a Picomolar Affinity HER2 Binding Affibody Molecule. Cancer Res 2006; 66:4339-4348.
Rao P S, Tian X, Qin W, Aruva M R, Sauter E R, Thakur M L, Wickstrom E. 99mTc-peptide-peptide nucleic acid probes for imaging oncogene mRNAs in tumours. Nucl Med Commun. 2003 August; 24(8):857-863.
Shim, H. Bispecific Antibodies and Antibody-Drug Conjugates for Cancer Therapy: Technological Considerations. Biomolecules 2020; 10:360.
Ståhl S, Gräslund T, Eriksson Karlström, A, Frejd F Y, Nygren P. Å, Löfblom J. Affibody molecules in biotechnological and medical applications. Trends Biotechnol. 2017; 35:691-712.
Sörensen J, Sandberg D, Sandström M, Wennborg A, Feldwisch J, Tolmachev V, Åström G, Lubberink M, Garske-Román U, Carlsson J, Lindman H. First-in-human molecular imaging of HER2 expression in breast cancer metastases using the 111In-ABY-025 affibody molecule. J Nucl Med. 2014 May; 55(5):730-735
Sörensen J, Velikyan I, Sandberg D, Wennborg A, Feldwisch J, Tolmachev V, Orlova A, Sandström M, Lubberink M, Olofsson H, Carlsson J, Lindman H. Measuring HER2-Receptor Expression In Metastatic Breast Cancer Using [68Ga]ABY-025 Affibody PET/CT. Theranostics. 2016 Jan. 1; 6(2):262-271.
Stickney D R, Anderson L D, Slater J B, Ahlem C N, Kirk G A, Schweighardt S A, Frincke J M. Bifunctional Antibody: A Binary Radiopharmaceutical Delivery System for Imaging Colorectal Carcinoma. Cancer Res Dec. 15 1991; (51)(24):6650-6655.
Tolmachev V, Orlova A. Affibody Molecules as targeting vectors for PET imaging. Cancers (Basel). 2020; 12(3):651.
Vorobyeva A, Westerlund K, Mitran B, et al. Development of an optimal imaging strategy for selection of patients for affibody-based PNA mediated radionuclide therapy. Scientific Reports. 2018; 8:9643.
Weidle U H, Auer J, Brinkmann U, Georges G, Tiefenthaler G. The Emerging Role of New Protein Scaffold-based Agents for Treatment of Cancer. Cancer Genomics Proteomics July-August 2013; 10(4):155-168.
Westerlund K, Honarvar H, Tolmachev V, Eriksson Karlström A Design, Preparation, and Characterization of PNA-Based Hybridization Probes for Affibody-Molecule-Mediated Pretargeting. Bioconjug Chem. 2015; 26:1724-1736.
Westerlund K, Altai M, Mitran B, et al. Radionuclide therapy of HER2-expressing human xenografts using an affibody molecule-based PNA-mediated pretargeting: in vivo proof-of-principle. J Nucl Med. 2018; 59:1092-1098.
Wållberg H, Orlova A. Slow internalization of anti-HER2 synthetic affibody monomer ¹¹¹In-DOTA-Z_{HER2:342}-pep2: implications for development of labeled tracers. Cancer Biother. Radiopharm. 2008; 23:435-442.
Zhao X, Wu C. Recent Advances in Peptide Nucleic Acids for Rapid Detection of Foodborne Pathogens. Food Anal. Methods. 2020; 13:1956-1972.

TABLE 1A
PNA probe sequences.

| | | | Mw |
|---|---|---|---|
| Name | Sequence | Reference | [Da] |
| HP1 | G-G-G-S-S-a-g-t-c-t-g-g-a-t-g-t-a-g-t-c-E-K | Westerlund et al, 2015 | 5396 |
| | (DOTA)-AEEA-E-NH₂ | | |
| HP2 | DOTA-AEEA-S-S-g-a-c-t-a-c-a-t-c-c-a-g-a-c-t- | Westerlund et al, 2015 | 5158 |
| | E-E-Y-NH₂ | | |
| HP15 | G-S-S-c-c-t-g-g-t-g-t-t-g-a-t-g-a-t-E-K(DOTA)- | | 5273 |
| | AEEA-E-NH₂ | | |
| HP16 | DOTA-AEEA-S-S-a-a-c-a-c-c-a-g-g-E-E-Y-NH₂ | | 3582 |
| HP17 | DOTA-AEEA-S-S-a-t-c-a-a-c-a-c-c-a-g-g-E-E-Y-NH₂ | | 4375 |
| HP18 | DOTA-AEEA-S-S-a-t-c-a-t-c-a-a-c-a-c-c-a-g-g-E- | | 5167 |
| | E-Y-NH₂ | | |
| HP19 | DOTA-AEEA-S-S-a-c-c-a-g-g-E-E-Y-NH₂ | | 2781 |

Amino acids are denoted by uppercase letters and PNA monomers are denoted by lowercase letters.
DOTA is the chelator 1,4,7,10-tetraazacyclododecane-1,4,7,10-tetraacetic acid.
AEEA is 2-[2-(2-aminoethoxy)ethoxy]acetic acid (also referred to as NH₂-(PEG)₂-CH₂COOH or 8-amino-3,6-dioxaoctanoic acid).

TABLE 1B
PNA sequences used in the probes shown in
Table 1A above.

| Name | PNA Sequence | SEQ ID NO: |
|---|---|---|
| HP1 | a-g-t-c-t-g-g-a-t-g-t-a-g-t-c | 1 |
| HP2 | g-a-c-t-a-c-a-t-c-c-a-g-a-c-t | 2 |
| HP15 | c-c-t-g-g-t-g-t-t-g-a-t-g-a-t | 3 |
| HP16 | a-a-c-a-c-c-a-g-g | 4 |
| HP17 | a-t-c-a-a-c-a-c-c-a-g-g | 5 |
| HP18 | a-t-c-a-t-c-a-a-c-a-c-c-a-g-g | 6 |
| HP19 | a-c-c-a-g-g | 7 |

TABLE 2
Kinetic parameters and melting temperatures for the secondary PNA
probes HP16, HP17, HP18 and HP19 binding to Z_{HER2:342}-SR-HP 15.

| | K_{D} (pM) | kₐ (M⁻¹ s⁻¹) | k_{d} (s⁻¹) | Tm (° C.) |
|---|---|---|---|---|
| HP16 | 280 | 4.6 x 10⁴ | 1.2 × 10⁻⁵ | 73 |
| HP17 | n.d. | 4.3 x 10⁴ | n.d. | 75 |
| HP18 | n.d. | 5.7 x 10⁴ | n.d. | 87 |
| HP19 | 3400 | 2.1 x 10⁷ | 7.2 × 10⁻² | 55 |

n.d. = Not determined

TABLE 3
¹⁷⁷Lu-labeling of PNA-based probes and in vitro stability.

| | | Radio- | | | |
|---|---|---|---|---|---|
| | | chemical | | | Stability |
| | | yield after | | Radio- | (×500 |
| | Radio- | EDTA | Isolated | chemical | EDTA), |
| | chemical | treatment, | yield, | purity, | 37° C., |
| | yield, % | % | % | % | 1 h |
| Z_{HER2:342}- | 78.5 ± 6.4 | 65.0 ± 7.1 | 66.0 ± 1.4 | 100 ± 0 | 100 ± 0 |
| SR-HP:15 | | | | | |
| HP16 | 99.8 ± 0.3 | 99.2 ± 1.4 | — | 99.2 ± 1.4 | 100 ± 0 |
| HP17 | 99.0 ± 1.7 | 98.8 ± 2.0 | — | 98.8 ± 2.0 | 100 ± 0 |
| HP18 | 97.5 ± 2.0 | 98.4 ± 2.7 | — | 98.4 ± 2.7 | 100 ± 0 |
| HP2 | 73.2 ± 0.2 | 69.6 ± 2.6 | 51.5 ± 9.2 | 100 ± 0 | — |

TABLE 4
Biodistribution of new [¹⁷⁷Lu]Lu-secondary probes in BALB/C nu/nu
mice bearing SKOV3 xenografts at 4 h post-injection (p.i.) without
pre-injection of primary agent. The uptake is expressed as % ID/g and
presented as an average value from 5 mice ± SD. One-way ANOVA with
Bonferroni's multiple comparisons test was performed to find
significant differences.

| | Uptake, % ID/g | | |
|---|---|---|---|
| | [¹⁷⁷Lu]-HP16 | [¹⁷⁷Lu]-HP17 | [¹⁷⁷Lu]-HP18 |
| Blood | 0.007 ± 0.003 | 0.005 ± 0.001 ^{c} | 0.019 ± 0.006 ^{b} |
| Lung | 0.09 ± 0.02 | 0.07 ± 0.01 ^{c} | 0.12 ± 0.02 |
| Liver | 0.08 ± 0.01 | 0.08 ± 0.02 ^{c} | 0.11 ± 0.02 ^{b} |
| Spleen | 0.053 ± 0.008 | 0.06 ± 0.01 | 0.08 ± 0.02 |
| Kidney | 3.6 ± 0.6 | 4.5 ± 0.4 ^{c} | 9.1 ± 0.9 ^{b} |
| Tumor | 0.12 ± 0.03 | 0.08 ± 0.01 | 0.10 ± 0.01 |
| Muscle | 0.025 ± 0.004 | 0.023 ± 0.006 | 0.029 ± 0.003 |
| Bone | 0.051 ± 0.008 ^{a} | 0.036 ± 0.007 ^{c} | 0.07 ± 0.02 |
| GI * | 0.29 ± 0.06 | 0.86 ± 0.21 | 0.65 ± 0.34 |
| Carcass * | 1.0 ± 0.4 | 1.18 ± 0.43 | 1.17 ± 0.17 |

^{a} significant difference between [¹⁷⁷Lu]-HP16 and [¹⁷⁷Lu]-HP17
^{b} significant difference between [¹⁷⁷Lu]-HP16 and [¹⁷⁷Lu]-HP18
^{c} significant difference between [¹⁷⁷Lu]-HP17 and [¹⁷⁷Lu]-HP18
* data for GI tract with content and carcass are presented as % of injected dose per whole sample.

TABLE 5
Biodistribution comparison of new [¹⁷⁷Lu]Lu-secondary probes in BALB/C nu/nu mice
bearing SKOV3 xenografts at 4 and 144 h after injection. Primary agents (Z_{HER2:342}-HP1
and Z_{HER2:342}-HP15, 50 μg) were injected 16 h prior to [¹⁷⁷Lu]Lu-secondary probe
injection. The uptake is expressed as % ID/g and presented as an average value from 5
mice ± SD. One-way ANOVA with Bonferroni's multiple comparisons test was
performed to find significant differences.

| | Uptake, % ID/g | | | | | | | |
|---|---|---|---|---|---|---|---|---|
| | 4 h | | | | 144 h | | | |
| | HP16 | HP17 | HP18 | HP2 | HP16 | HP17 | HP18 | HP2 |
| Blood | 0.04 ± 0.01ª | 0.05 ± 0.01^{b} | 0.17 ± 0.02^{a, b, c} | 0.049 ± 0.004^{c} | NM | NM | NM | NM |
| Lung | 0.16 ± 0.03^{d} | 0.17 ± 0.04^{b} | 0.30 ± 0.03^{b, c, d} | 0.19 ± 0.03^{c} | NM | NM | NM | NM |
| Liver | 0.10 ± 0.02^{a} | 0.11 ± 0.01^{b} | 0.2 ± 0.1^{a, b, c} | 0.11 ± 0.02^{c} | 0.014 ± 0.003^{a} | 0.010 ± 0.004^{b} | 0.07 ± 0.01^{a, b, c} | 0.015 ± 0.003^{c} |
| Spleen | 0.07 ± 0.01^{a} | 0.09 ± 0.03 | 0.12 ± 0.02^{a} | 0.08 ± 0.03 | NM | NM | NM | NM |
| Kidney | 6 ± 1^{a, d} | 8 ± 1^{b} | 12 ± 2^{a, b} | 10 ± 24 | 0.7 ± 0.2^{a} | 0.67 ± 0.05^{b} | 1.6 ± 0.5^{a,b,c} | 0.9 ± 0.2^{c} |
| Tumor | 24 ± 6 | 22 ± 3 | 22 ± 1 | 19 ± 1 | 3 ± 1^{a} | 5 ± 1 | 5 ± 1^{a} | 4 ± 1 |
| Muscle | 0.06 ± 0.02 | 0.06 ± 0.02 | 0.07 ± 0.03 | 0.04 ± 0.01 | NM | NM | NM | NM |
| Bone | 0.06 ± 0.02^{a} | 0.07 ± 0.01 | 0.10 ± 0.02^{a, c} | 0.08 ± 0.06^{c} | NM | NM | NM | NM |
| GI | 0.4 ± 0.1 | 0.5 ± 0.3 | 0.4 ± 0.2 | 0.4 ± 0.1 | 0.02 ± 0.01 | 0.05 ± 0.02 | 0.08 ± 0.06 | 0.09 ± 0.07 |
| Carcass | 0.7 ± 0.1 | 1.5 ± 0.9 | 1.8 ± 0.9 | 1.6 ± 0.7 | 0.09 ± 0.02 | 0.12 ± 0.07 | 0.24 ± 0.02 | 0.2 ± 0.1 |

^{a}significant difference (p < 0.05) between [¹⁷⁷Lu]Lu-HP16 and [¹⁷⁷Lu]Lu-HP18
^{b}significant difference (p < 0.05) between [¹⁷⁷Lu]Lu-HP17 and [¹⁷⁷Lu]Lu-HP18
^{c}significant difference (p < 0.05) between [¹⁷⁷Lu]Lu-HP18 and [¹⁷⁷Lu]Lu-HP2
^{d}significant difference (p < 0.05) between [¹⁷⁷Lu]Lu-HP16 and [¹⁷⁷Lu]Lu-HP2
NM = Not measured

TABLE 6
Areas under time-activity plot.

| | | | Tumor-to-kidney |
|---|---|---|---|
| | Area for kidneys | Area for tumor | ratio |
| [¹⁷⁷Lu]Lu-HP16 | 4.6 | 17.8 | 3.8 |
| [¹⁷⁷Lu]Lu-HP17 | 6.1 | 17.3 | 2.8 |
| [¹⁷⁷Lu]Lu-HP18 | 8.9 | 17.3 | 2.0 |
| [¹⁷⁷Lu]Lu-HP2 | 7.3 | 14.7 | 2.0 |

TABLE 7
Examples of proteins that can be targeted according to the invention.
In addition to the specified human proteins, mammalian orthologs
are also included for use according to the invention. References to the
UniProt database (www.uniprot.org) are valid as of Oct. 12, 2020.

| | UniProt reference to human |
|---|---|
| Protein | protein |
| Epidermal growth factor receptor (EGFR) | P00533 (EGFR_HUMAN) |
| Human epidermal growth factor receptor 2 | P04626 (ERBB2_HUMAN) |
| (HER2) | |
| Human epidermal growth factor receptor 3 | P21860 (ERBB3_HUMAN) |
| (HER3) | |
| Insulin-like growth factor 1 receptor | P08069 (IGF1R_HUMAN) |
| (IGF1R) | |
| Carbonic anhydrase IX (CAIX) | Q16790 (CAH9_HUMAN) |
| Platelet-derived growth factor receptor β | P09619 (PGFRB_HUMAN) |
| (PDGFR-β) | |
| Nectin-4 | Q96NY8 (NECT4_HUMAN) |
| Cluster of differentiation 38 (CD38) | P28907 (CD38_HUMAN) |
| Cluster of differentiation 33 (CD33) | P20138 (CD33_HUMAN) |
| Cluster of differentiation 30 (CD30) | P28908 (TNR8_HUMAN) |
| Cluster of differentiation 22 (CD22) | P20273 (CD22_HUMAN) |
| Cluster of differentiation 79b (CD79b) | P40259 (CD79B_HUMAN) |

1. A kit for targeting of a diagnostic or therapeutic agent to a target site comprising:
(a) a first conjugate comprising
(i) a targeting moiety capable of binding selectively to the target site; and
(ii) a first hybridization probe moiety comprising a first PNA oligomer; and
(b) a second conjugate comprising
(i) a second hybridization probe moiety comprising a second PNA oligomer complementary to the first PNA oligomer; and
(ii) a diagnostic agent or a therapeutic agent moiety;
wherein the length of the second PNA oligomer in the second hybridization probe is between 5 and 12 bases.

2. The kit according to claim 1 wherein the said target site resides on a mammalian, including human, protein which is expressed on the surface of a cell.

3. The kit according to claim 2 wherein the said target site resides on a mammalian, including human, protein selected from the group consisting of epidermal growth factor receptor (EGFR), human epidermal growth factor receptor 2 (HER2), human epidermal growth factor receptor 3 (HER3), insulin-like growth factor 1 receptor (IGF1R), carbonic anhydrase IX (CAIX), platelet-derived growth factor receptor β (PDGFR-β), nectin-4, cluster of differentiation 38 (CD38), cluster of differentiation 33 (CD33), cluster of differentiation 30 (CD30), cluster of differentiation 22 (CD22), and cluster of differentiation 79b (CD79b).

4. The kit according to claim 3 wherein the said target site resides on human epidermal growth factor receptor 2 (HER2).

5. The kit according to claim 1 wherein the said targeting moiety is selected from the group consisting of:
antibodies;
antibody fragments, single-chain variable fragments (scFv), antigen-binding fragments (Fab), diabodies, or minibodies;
engineered scaffold proteins; and
peptides.

6. (canceled)

7. The kit according to claim 1 wherein one or both of the hybridization probes comprise a solubilizing moiety; or a peptide-based linker comprising charged or polar amino acids.

8. The kit according to claim 1 wherein the length of the first PNA oligomer in the first hybridization probe moiety is at least the length of the second PNA oligomer in the second hybridization probe moiety, and not more than 15 bases.

9. (canceled)

10. The kit according to claim 1 wherein the length of said complementary PNA oligomer in the second hybridization probe moiety is 8-12 bases.

11. The kit according to claim 10 wherein the length of said complementary PNA oligomer is 9 bases.

12. The kit according to claim 10 wherein the length of said complementary PNA oligomer is 8 bases.

13. The kit according to claim 1 wherein the said therapeutic agent is selected from the group consisting of radionuclides, calicheamicin, auristatins, and maytansinoids.

14. The kit according to claim 13 wherein the said therapeutic agent is a radionuclide selected from the group consisting of Lutetium-177 (¹⁷⁷Lu), Yttrium-90 (⁹⁰Y), Bismuth-212 (²¹²Bi) Bismuth-213 (²¹³Bi), Astatine-211 (²¹¹At), Actinium-255 (²⁵⁵Ac), Copper-67 (⁶⁷Cu), Gallium-67 (⁶⁷Ga), and Rhenium-186 (¹⁸⁶Re).

15. The kit according to claim 1 wherein the said diagnostic agent generates a signal that is detectable by a method selected from the group consisting of magnetic resonance imaging (MM), positron emission tomography (PET), single photon emission computed tomography (SPECT), computed tomography (CT), X-ray imaging, ultrasound, and optical imaging.

16. The kit according to claim 15 wherein the said diagnostic agent is a radionuclide.

17. (canceled)

18. A method for delivering a diagnostic or therapeutic agent to a target site in a mammal, including humans, said method comprising:
(a) administering to said mammal a first conjugate comprising
(i) a targeting moiety which binds selectively to the target site; and
(ii) a first hybridization probe moiety comprising a first PNA oligomer;
(b) optionally, administering to said mammal a clearing agent, and allowing said clearing agent to clear non-localized first conjugate from circulation; and
(c) administering to said mammal a second conjugate comprising
(i) a second hybridization probe moiety comprising a second PNA oligomer complementary to the first PNA oligomer; and
(ii) a diagnostic agent or a therapeutic agent moiety;
wherein the length of the second PNA oligomer in the second hybridization probe moiety is between 5 and 12 bases, and wherein said second PNA oligomer binds to the first PNA oligomer in the first conjugate, thereby targeting the diagnostic agent or therapeutic agent moiety to the target site.

19. A pharmaceutical composition comprising the first and second conjugates of a kit according to claim 1.

20. A method of diagnosis or treatment of a medical condition selected from the group consisting of cancer, infectious diseases, inflammatory diseases, and autoimmune diseases in a subject, said method comprising administering to the subject the pharmaceutical composition according to claim 19.

21. (canceled)

22. The method according to claim 18, which is a method for the diagnosis, prognosis or treatment of a medical condition selected from the group consisting of cancer, infectious diseases, inflammatory diseases, and autoimmune diseases.

23. (canceled)

24. The method according to claim 22, wherein said medical condition is cancer capable of forming solid tumors, said cancer being selected from the group consisting of breast cancer, prostate cancer, lung cancer, head- and neck cancer, gastric cancer, and colon cancer.

25-28. (canceled)

29. The kit according to claim 2 wherein the said target site resides on a mammalian, including human, protein which is expressed on the surface of a tumor cell.


SEQUENCE LISTING (7 sequences)

SEQ ID NO: 1 - DNA, 15 bp, Artificial Sequence
  Synthetic PNA sequence
agtctggatgtagtc

SEQ ID NO: 2 - DNA, 15 bp, Artificial Sequence
  Synthetic PNA sequence
gactacatccagact

SEQ ID NO: 3 - DNA, 15 bp, Artificial Sequence
  Synthetic PNA sequence
cctggtgttgatgat

SEQ ID NO: 4 - DNA, 9 bp, Artificial Sequence
  Synthetic PNA sequence
aacaccagg

SEQ ID NO: 5 - DNA, 12 bp, Artificial Sequence
  Synthetic PNA sequence
atcaacaccagg

SEQ ID NO: 6 - DNA, 15 bp, Artificial Sequence
  Synthetic PNA sequence
atcatcaacaccagg

SEQ ID NO: 7 - DNA, 6 bp, Artificial Sequence
  Synthetic PNA sequence
accagg